(19) United States
(12) Patent Application Publication
(10) Pub. No.: US 2019/0345495 A1
(43) Pub. Date: Nov. 14, 2019

(54) ANTISENSE OLIGONUCLEOTIDES FOR MODULATING NFKB2 EXPRESSION

(71) Applicant: Roche Innovation Center Copenhagen A/S, Hørsholm (DK)

(72) Inventors: Eva Marie W. LINDHOLM, Hørsholm (DK); Lykke PEDERSEN, Hørsholm (DK); Steffen SCHMIDT, Hørsholm (DK)

(21) Appl. No.: 16/477,854

(22) PCT Filed: Jan. 10, 2018
(86) PCT No.: PCT/EP2018/050584

§ 371 Date: Jul. 12, 2019

(30) Foreign Application Priority Data
Jan. 13, 2017 (EP) 17151404.5

(51) Int. Cl. C12N 15/113

(52) U.S. Cl. CPC C12N 15/113; C12N 2310/11; C12N 2310/315; C12N 2310/341; C12N 2310/3341; C12N 2310/3231

(57) ABSTRACT

The present invention relates to antisense oligonucleotides that are capable of modulating expression of NF-kB2 in a target cell. The oligonucleotides are complementary to mammalian NFKB2 pre-mRNA sequence. The present invention further relates to conjugates of the oligonucleotide and pharmaceutical compositions and methods for treatment of cancer, inflammation or autoimmune diseases using the oligonucleotide.

### FIELD OF INVENTION

The present invention relates to oligonucleotides (oligomers) complementary to NFKB2 pre-mRNA sequences, which are capable inhibiting the expression of NF-κB2. Inhibition of NF-κB2 expression is beneficial for a range of medical disorders including autoimmunity and cancer.

### BACKGROUND

Nuclear factor kappa-light-chain-enhancer of activated B cells (NF-κB) is a key regulator of processes such as immunity, inflammation, gene expression, cancer cell migration, invasion, apoptosis, and proliferation. NF-κB subunits share a Rel homology domain in their N-terminus. The NFKB2 (nuclear factor kappa B subunit 2) gene encodes a 100 kD protein (p100). In what is referred to as the “alternative pathway” for NF-κB signaling, p100 for is processed to a 52 kD protein (p52). p52 as homo- or heterodimer can form some members of the NF-κB family of transcription factors, specific for different tissues and subsets of cells.

NF-κB subunit expression can be altered in disease, and dysfunctional NF-κB activation contributes to disorders including rheumatoid arthritis, atherosclerosis, inflammatory bowel diseases, multiple sclerosis and malignant tumors (Park and Hong, 2016, Cells 5:15), as well as in asthma and chronic inflammatory airway disease (Schuliga, 2015, Biomolecules, 5-1266). Aggravated activation of the alternative pathway of NF-κB signaling has been correlated with disorders like rheumatoid arthritis, ulcerative colitis or B cell lymphomas suggesting that p52 reduction can be an option for treatment of inflammatory disorders and cancers (Dejardin, 2006, Biochem Pharm 72: 1161).

There are >700 compounds described in literature to have NF-κB inhibitory effect, most of them with broad effect on NF-κB signaling, but a narrow therapeutic index, poor specificity, short in vivo half-life of molecules, and only minor effects on signaling, and have therefore limited the therapeutic use of described NF-κB inhibitors to date. It has been suggested that specific NF-κB subunit reduction can be a way to overcome the limitations of more “general” NF-κB inhibitors.

Ishige et al, Neurochemistry 47, 545-55 reports that distinct nuclear factor-kB/Rel proteins have opposing modulatory effects in glutamate-induced cell death in HT22 cells, and discloses antisense oligonucleotides targeting NF-κB subunits, including a compound of sequence 5′-TCGTAGCAATTGTCCATATCT-3′ which apparent targets p52.

WO00/31111 discloses numerous phosphorothioate oligodeoxynucleotides targeting human p65 (see table 1), including 2′-MOE gapmers (table 2). One of these compounds, ISIS 23810, has partial complementarity to human NFKB2.

### OBJECTIVE OF THE INVENTION

The present invention identifies novel oligonucleotides which inhibit human NFKB2 which are useful in the treatment of a range of medical disorders including autoimmunity, inflammation and cancer.

### SUMMARY OF INVENTION

The present invention relates to oligonucleotides targeting a NFKB2 nucleic acid, capable of modulating, such as inhibiting the expression of NF-κB2.

The invention provides for an antisense oligonucleotide of 10 to 30 contiguous nucleotides in length, wherein at least 10 contiguous nucleotides of the contiguous sequence of the oligonucleotide is at least 90% complementarity, such as fully complementary, to a NFKB2 sequence.

The invention provides for an LNA antisense oligonucleotide of 10 to 30 contiguous nucleotides in length, wherein at least 10 contiguous nucleotides of the contiguous sequence of the oligonucleotide is at least 90% complementarity, such as fully complementary, to a NFKB2 sequence.

The invention provides for an LNA antisense oligonucleotide of 10 to 30 contiguous nucleotides in length, wherein at least 14 contiguous nucleotides of the contiguous sequence of the oligonucleotide is fully complementary, to the NFKB2 pre-mRNA (SEQ ID NO 21).

The invention provides for an LNA antisense oligonucleotide, which is capable of inhibiting NFKB2 expression in a cell which is expressing NF-κB2, which consists or comprises of a contiguous nucleotide sequence of 10 to 30 nucleotides in length with at least 90% complementarity, such as fully complementarity, to a NFKB2 sequence.

The invention provides for an antisense oligonucleotide of 12 to 30 contiguous nucleotides in length, wherein at least 10 contiguous nucleotides of the contiguous sequence of the oligonucleotide is at least 90% complementarity, such as fully complementary, to a NFKB2 sequence.

The invention provides for an LNA antisense oligonucleotide of 12 to 30 contiguous nucleotides in length, wherein at least 10 contiguous nucleotides of the contiguous sequence of the oligonucleotide is at least 90% complementarity, such as fully complementary, to a NFKB2 sequence.

The invention provides for an LNA antisense oligonucleotide of 12 to 30 contiguous nucleotides in length, wherein at least 14 contiguous nucleotides of the contiguous sequence of the oligonucleotide is fully complementary, to the NFKB2 pre-mRNA (SEQ ID NO 21).

The invention provides for an LNA antisense oligonucleotide, which is capable of inhibiting NF-κB2 expression in a cell which is expressing NFKB2, which consists or comprises of a contiguous nucleotide sequence of 12 to 30 nucleotides in length with at least 90% complementarity, such as fully complementarity, to a NFKB2 sequence.

The invention provides for a conjugate comprising the oligonucleotide according to the invention.

In a further aspect, the invention provides pharmaceutical compositions comprising the oligonucleotide or conjugate of the invention and pharmaceutically acceptable diluents, carriers, salts and/or adjuvants.

In a further aspect, the invention provides methods for in vivo or in vitro method for modulation of NF-κB2 expression in a target cell which is expressing NFKB2 by administering an oligonucleotide, conjugate, or composition of the invention in an effective amount to said cell.

In a further aspect the invention provides methods for treating or preventing a disease, disorder or dysfunction associated with in vivo activity of NF-κB2 comprising administering a therapeutically or prophylactically effective amount of the oligonucleotide, conjugate or composition of the invention to a subject suffering from or susceptible to the disease, disorder or dysfunction.

In a further aspect the oligonucleotide, conjugate or composition of the invention is used for the treatment or prevention of cancer, autoimmune diseases, and inflammation or an inflammatory disease.

In some embodiments, the oligonucleotide, conjugate or composition of the invention is an antisense oligonucleotides, preferably a gapmer antisense oligonucleotide.

### BRIEF DESCRIPTION OF FIGURES

FIG. 1A, 1B and 1C: Mouse in vivo efficacy, 16 days of treatment, intravenous injection (tail vein).

FIG. 2: Testing in vitro efficacy of various antisense oligonucleotides targeting human NFKB2 mRNA in HEK293 and HeLa cell lines at single dose concentration.

FIG. 3: Testing in vitro efficacy of antisense oligonucleotides targeting human NFKB2 mRNA in HEK293 and HeLa cell lines at single dose concentration.

FIG. 4: Testing in vitro efficacy of antisense oligonucleotides targeting human NFKB2 mRNA in HEK293 and HeLa cell lines at single dose concentration. Zoom in illustrating the data for compounds targeting the hot spot regions.

FIG. 5A, 5B & 5C: Testing in vitro potency and efficacy of selected oligonucleotides targeting human NFKB2 mRNA in HEK-293 and HeLa cell lines in a dose response curve.

FIG. 6: Human NFKB2 pre-mRNA sequence (SEQ ID NO 21) derived from the human genomic sequence NC_000010.11 (102394110 . . . 102402529).

### DEFINITIONS

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

Antisense Oligonucleotides

The term “Antisense oligonucleotide” as used herein is defined as oligonucleotides capable of modulating expression of a target gene by hybridizing to a target nucleic acid, in particular to a contiguous sequence (a sub-sequence) on a target nucleic acid. The antisense oligonucleotides are not essentially double stranded and are therefore not siRNAs. Preferably, the antisense oligonucleotides of the present invention are single stranded.

An LNA antisense oligonucleotide is an antisense oligonucleotide which comprises at least one LNA nucleoside. In some embodiments the LNA antisense oligonucleotide is a LNA gapmer oligonucleotide.

Targeting

The oligonucleotides of the invention are capable of targeting the human NFKB2 transcript. Targeting refers to the ability of the oligonucleotide to form a functional complementary hybridization across the contiguous nucleotide sequence of the oligonucleotide with the human NFKB2 transcript, such as a fully complementary hybridization, and inhibit the expression of the human NFKB2 transcript in a cell.

Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence and may optionally comprise further nucleotide(s), for example a nucleotide linker region which may be used to attach a functional group to the contiguous nucleotide sequence. The nucleotide linker region may or may not be complementary to the target nucleic acid.

Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In some embodiments the modified nucleoside comprises a modified sugar moiety. The term modified nucleoside may also be used herein interchangeably with the term “nucleoside analogue” or modified “units” or modified “monomers”.

Modified Internucleoside Linkage

The term “modified internucleoside linkage” is defined as generally understood by the skilled person as linkages other than phosphodiester (PO) linkages, that covalently couples two nucleosides together. Nucleotides with modified internucleoside linkage are also termed “modified nucleotides”. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides.

In an embodiment, the oligonucleotide comprises one or more internucleoside linkages modified from the natural phosphodiester to a linkage that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are modified. In some embodiments all of the internucleoside linkages of the oligonucleotide, or contiguous nucleotide sequence thereof, are modified. It will be recognized that, in some embodiments the nucleosides which link the oligonucleotide of the invention to a non-nucleotide functional group, such as a conjugate, may be phosphodiester. In some embodiments all of the internucleoside linkages of the oligonucleotide, or contiguous nucleotide sequence thereof, are nuclease resistant internucleoside linkages.

Modified internucleoside linkages may be selected from the group comprising phosphorothioate, diphosphorothioate and boranophosphate. In some embodiments, the modified internucleoside linkages are compatible with the RNaseH recruitment of the oligonucleotide of the invention, for example phosphorothioate, diphosphorothioate or boranophosphate.

In some embodiments the internucleoside linkage comprises sulphur (S), such as a phosphorothioate internucleoside linkage.

A phosphorothioate internucleoside linkage is particularly useful due to nuclease resistance, beneficial pharmakokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80 or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

In some embodiments, the oligonucleotide comprises one or more neutral internucleoside linkage, particularly a internucleoside linkage selected from phosphotriester, methylphosphonate, MMI, amide-3, formacetal or thioformacetal.

Further internucleoside linkages are disclosed in WO2009/124238 (incorporated herein by reference). In an embodiment the internucleoside linkage is selected from linkers disclosed in WO2007/031091 (incorporated herein by reference). Particularly, the internucleoside linkage may be selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, —NR^{H}—CO—NR^{H}—, and/or the internucleoside linker may be selected form the group consisting of: —O—Co—O—, —O—CO—NR^{H}—, —NR^{H}—CO—CH₂—, —O—CH₂—CO—NR^{H}—, —O—CH₂—CH₂—NR^{H}—, —CO—NR^{H}—CH₂—, —CH₂—NR^{H}CO—, —O—CH₂—CH₂—S—, —S—CH₂—CH₂—O—, —S—CH₂—CH₂—S—, —CH₂—SO₂—CH₂—, —CH₂—CO—NR^{H}—, —O—CH₂—CH₂—NR^{H}—CO—CH₂—NCH₃—O—CH₂—, where R^{H} is selected from hydrogen and C₁₋₄ alkyl.

Nuclease resistant linkages, such as phosphothioate linkages, are particularly useful in oligonucleotide regions capable of recruiting nuclease when forming a duplex with the target nucleic acid, such as region G for gapmers, or the non-modified nucleoside region of headmers and tailmers. Phosphorothioate linkages may, however, also be useful in non-nuclease recruiting regions and/or affinity enhancing regions such as regions F and F′ for gapmers, or the modified nucleoside region of headmers and tailmers.

Each of the design regions may however comprise internucleoside linkages other than phosphorothioate, such as phosphodiester linkages, in particularly in regions where modified nucleosides, such as LNA, protect the linkage against nuclease degradation. Inclusion of phosphodiester linkages, such as one or two linkages, particularly between or adjacent to modified nucleoside units (typically in the non-nuclease recruiting regions) can modify the bioavailability and/or bio-distribution of an oligonucleotide—see WO2008/113832, incorporated herein by reference.

In an embodiment all the internucleoside linkages in the oligonucleotide are phosphorothioate and/or boranophosphate linkages. In some embodiments, all the internucleoside linkages in the oligonucleotide are phosphorothioate linkages.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

Complementarity

The term “complementarity” describes the capacity for Watson-Crick base-pairing of nucleosides/nucleotides. Watson-Crick base pairs are guanine (G)-cytosine (C) and adenine (A)—thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al. (2012) Accounts of Chemical Research vol 45 page 2055 and Bergstrom (2009) Current Protocols in Nucleic Acid Chemistry Suppl. 37 1.4.1).

The term “% complementary” as used herein, refers to the number of nucleotides in percent of a contiguous nucleotide sequence in a nucleic acid molecule (e.g. oligonucleotide) which, at a given position, are complementary to (i.e. form Watson Crick base pairs with) a contiguous nucleotide sequence, at a given position of a separate nucleic acid molecule (e.g. the target nucleic acid). The percentage is calculated by counting the number of aligned bases that form pairs between the two sequences, dividing by the total number of nucleotides in the oligonucleotide and multiplying by 100. In such a comparison a nucleobase/nucleotide which does not align (form a base pair) is termed a mismatch.

The term “fully complementary”, refers to 100% complementarity.

Identity

The term “Identity” as used herein, refers to the number of nucleotides in percent of a contiguous nucleotide sequence in a nucleic acid molecule (e.g. oligonucleotide) which, at a given position, are identical to (i.e. in their ability to form Watson Crick base pairs with the complementary nucleoside) a contiguous nucleotide sequence, at a given position of a separate nucleic acid molecule (e.g. the target nucleic acid). The percentage is calculated by counting the number of aligned bases that are identical between the two sequences, including gaps, dividing by the total number of nucleotides in the oligonucleotide and multiplying by 100.

Percent Identity=(Matches×100)/Length of aligned region (with gaps).

Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ, is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965,Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG° . The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or —16 to —27 kcal such as —18 to —25 kcal.

Target Nucleic Acid

According to the present invention, the target nucleic acid is a nucleic acid which encodes mammalian NF-κB2 and may for example be a gene, a RNA, a mRNA, and pre-mRNA, a mature mRNA or a cDNA sequence. The target may therefore be referred to as an NFKB2 target nucleic acid.

For in vivo or in vitro application, the oligonucleotide of the invention is typically capable of inhibiting the expression of the NFKB2 target nucleic acid in a cell which is expressing the NFKB2 target nucleic acid. The contiguous sequence of nucleobases of the oligonucleotide of the invention is typically complementary to the NF-κB2 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″). The target nucleic acid may, in some embodiments, be a NFKB2 pre-mRNA

Target Sequence

The term “target sequence” as used herein refers to a sequence of nucleotides present in the target nucleic acid which comprises the nucleobase sequence which is complementary to the oligonucleotide of the invention. In some embodiments, the target sequence consists of a region on the target nucleic acid which is complementary to the contiguous nucleotide sequence of the oligonucleotide of the invention. In some embodiments the target sequence is longer than the complementary sequence of a single oligonucleotide, and may, for example represent a preferred region of the target nucleic acid which may be targeted by several oligonucleotides of the invention.

The target sequence may be a sub-sequence of the target nucleic acid.

In some embodiments the sub-sequence is a sequence selected from the group consisting of SEQ ID NO 11, 12, 13, 14, 15 or 16, 17, 18, 19 and 20.

The oligonucleotide of the invention comprises a contiguous nucleotide sequence which is complementary to or hybridizes to the target nucleic acid, such as a sub-sequence of the target nucleic acid, such as a target sequence described herein.

The oligonucleotide comprises a contiguous nucleotide sequence of at least 8 nucleotides which is complementary to or hybridizes to a target sequence present in the target nucleic acid molecule. The contiguous nucleotide sequence (and therefore the target sequence) comprises of at least 8 contiguous nucleotides, such as 9, 10, 11, 12, 13, 14, 15, 16, 17, 18, 19, 20, 21, 22, 23, 24, 25, 26, 27, 28, 29 or 30 contiguous nucleotides, such as from 12-25, such as from 14-18 contiguous nucleotides.

Target Cell

The term a “target cell” as used herein refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell or a human cell.

In preferred embodiments the target cell expresses NFKB2 pre-mRNA.

In some embodiments the oligonucleotides, conjugates or compositions, of the invention are capable to inhibiting the expression of human NF-κB2 in a cell selected from the group consisting of HEK-293 and HeLa cells.

Naturally Occurring Variant

The term “naturally occurring variant” refers to variants of NFKB2 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms, and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95% such as at least 98% or at least 99% homology to a mammalian NFKB2 target nucleic acid, such SEQ ID NO 21.

Modulation of Expression

The term “modulation of expression” as used herein is to be understood as an overall term for an oligonucleotide's ability to alter the amount of NFKB2 when compared to the amount of NFKB2 before administration of the oligonucleotide. Alternatively modulation of expression may be determined by reference to a control experiment. It is generally understood that the control is an individual or target cell treated with a saline composition or an individual or target cell treated with a non-targeting oligonucleotide (mock). It may however also be an individual treated with the standard of care.

One type of modulation is an oligonucleotide's ability to inhibit, down-regulate, reduce, suppress, remove, stop, block, prevent, lessen, lower, avoid or terminate expression of NF-κB2 e.g. by degradation of mRNA or blockage of transcription.

High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between+3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

2′ Modified Nucleosides.

A 2′ sugar modified nucleoside is a nucleoside which has a substituent other than H or —OH at the 2′ position (2′ substituted nucleoside) or comprises a 2′ linked biradicle, and includes 2′ substituted nucleosides and LNA (2′-4′ biradicle bridged) nucleosides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

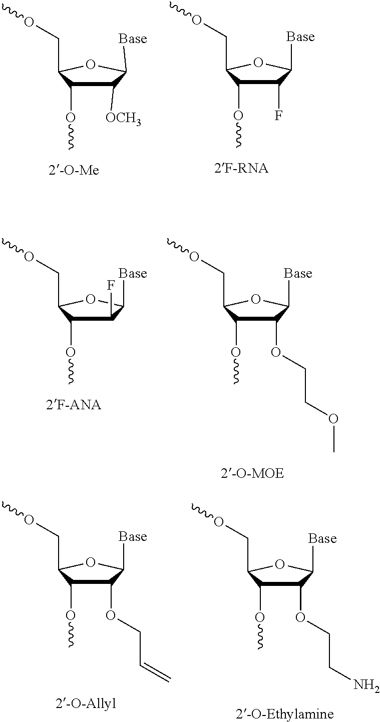

Locked Nucleic Acid Nucleosides (LNA).

LNA nucleosides are modified nucleosides which comprise a linker group (referred to as a biradicle or a bridge) between C2′ and C4′ of the ribose sugar ring of a nucleotide. These nucleosides are also termed bridged nucleic acid or bicyclic nucleic acid (BNA) in the literature.

In some embodiments, the modified nucleoside or the LNA nucleosides of the oligomer of the invention has a general structure of the formula I or II:

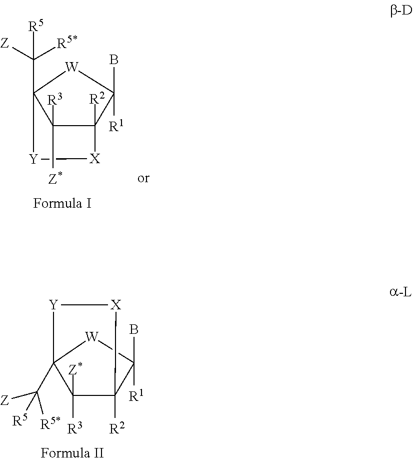

wherein W is selected from —O—, —S—, —N(R^{a})—, —C(R^{a}R^{b})—, such as, in some embodiments —O—;

B designates a nucleobase or modified nucleobase moiety;

Z designates an internucleoside linkage to an adjacent nucleoside, or a 5′-terminal group;

Z* designates an internucleoside linkage to an adjacent nucleoside, or a 3′-terminal group;

X designates a group selected from the list consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z
In some embodiments, X is selected from the group consisting of: —O—, —S—, NH—, NR^{a}R^{b}, —CH₂—, CR^{a}R^{b}, —C(═CH₂)—, and —O(═CR^{a}R^{b})—
In some embodiments, X is —O—

Y designates a group selected from the group consisting of —C(R¹R^{b})—, —C(R^{a})═C(R^{b})—, —C(R^{a})═N—, —O—, —Si(R^{a})₂—, —S—, —SO₂—, —N(R^{a})—, and >C═Z
In some embodiments, Y is selected from the group consisting of: —CH₂—, —C(R¹R^{b})—, —CH₂CH₂—, —C(R^{a}R^{b})—C(R^{a}R^{b})—, —CH₂CH₂CH₂—, —C(R¹R^{b})C(R¹R^{b})C(R¹R^{b})—, —C(R^{a})═C(R^{b})—, and —C(R^{a})═N—
In some embodiments, Y is selected from the group consisting of: —CH₂—, —CHR^{a}—, —CHCH₃—, CR^{a}R^{b}—
   or —X—Y— together designate a bivalent linker group (also referred to as a radicle) together designate a bivalent linker group consisting of 1, 2, 3 or 4 groups/atoms selected from the group consisting of —C(R¹R^{b})—, —C(R^{a})═C(R^{b})—, —C(R^{a})═N—, —O—, —Si(R^{a})₂—, —S—, —SO₂—, —N(R^{a})—, and >C═Z,
In some embodiments, —X—Y— designates a biradicle selected from the groups consisting of: —X—CH₂—, —X—CR^{a}R^{b}—, —X—CHR^{a}—, —X—C(HCH₃)—, —O—Y—, —O—CH₂—, —S—CH₂—, —NH—CH₂—, —O—CHCH₃—, —CH₂—O—OH₂, —O—CH(CH₃CH₃)—, —O—CH₂—CH₂—, OCH₂—OH₂—OH₂—,—O—CH₂OCH₂—, —O—NCH₂—, —O(═CH₂)—CH₂—, —NR^{a}—CH₂—, N—O—CH₂, —S—CR^{a}R^{b}— and —S—CHR^{a}—.
In some embodiments —X—Y— designates —O—CH₂- or —O—CH(0H₃)-.

wherein Z is selected from —O—, —S—, and —N(R^{a})—,

and R^{a} and, when present R^{b}, each is independently selected from hydrogen, optionally substituted C₁₋₆-alkyl, optionally substituted C₂₋₆-alkenyl, optionally substituted C₂₋₆-alkynyl, hydroxy, optionally substituted C₁₋₆-alkoxy, C₂₋₆-alkoxyalkyl, C₂₋₆-alkenyloxy, carboxy, C₁₋₆-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, where aryl and heteroaryl may be optionally substituted and where two geminal substituents R^{a} and R^{b} together may designate optionally substituted methylene (═CH₂), wherein for all chiral centers, asymmetric groups may be found in either R or S orientation.

wherein R¹, R², R³, R⁵ and R⁵* are independently selected from the group consisting of: hydrogen, optionally substituted C₁₋₆-alkyl, optionally substituted C₂₋₆-alkenyl, optionally substituted C₂₋₆-alkynyl, hydroxy, C₁₋₆-alkoxy, C₂₋₆-alkoxyalkyl, C₂₋₆-alkenyloxy, carboxy, C₁₋₆-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, carbonylamino, carbamido, C₁₋₆-alkanoyloxy, sulphono, C₁₋₆-alkylsulphonyloxy, nitro, azido, sulphanyl, C₁₋₆-alkylthio, halogen, where aryl and heteroaryl may be optionally substituted, and where two geminal substituents together may designate oxo, thioxo, imino, or optionally substituted methylene.
In some embodiments R¹, R², R³, R⁵ and R⁵* are independently selected from C₁₋₆ alkyl, such as methyl, and hydrogen.
In some embodiments R¹, R², R³, R⁵ and R⁵* are all hydrogen.
In some embodiments R¹, R², R³, are all hydrogen, and either R⁵ and R⁵* is also hydrogen and the other of R⁵ and R⁵* is other than hydrogen, such as C₁₋₆ alkyl such as methyl.
In some embodiments, R^{a} is either hydrogen or methyl. In some embodiments, when present, R^{b} is either hydrogen or methyl.
In some embodiments, one or both of R^{a} and R^{b} is hydrogen
In some embodiments, one of R^{a} and R^{b} is hydrogen and the other is other than hydrogen
In some embodiments, one of R^{a} and R^{b} is methyl and the other is hydrogen
In some embodiments, both of R^{a} and R^{b} are methyl.

In some embodiments, the biradicle —X—Y— is —O—CH₂—, W is O, and all of R¹, R², R³, R⁵ and R⁵* are all hydrogen. Such LNA nucleosides are disclosed in WO99/014226, WO00/66604, WO98/039352 and WO2004/046160 which are all hereby incorporated by reference, and include what are commonly known as beta-D-oxy LNA and alpha-L-oxy LNA nucleosides.

In some embodiments, the biradicle —X—Y— is —S—CH₂—, W is O, and all of R¹, R², R³, R⁵ and R⁵* are all hydrogen. Such thio LNA nucleosides are disclosed in WO99/014226 and WO2004/046160 which are hereby incorporated by reference.

In some embodiments, the biradicle —X—Y— is —NH—CH₂—, W is O, and all of R¹, R², R³, R⁵ and R⁵* are all hydrogen. Such amino LNA nucleosides are disclosed in WO99/014226 and WO2004/046160 which are hereby incorporated by reference.

In some embodiments, the biradicle —X—Y— is —O—CH₂—CH₂— or —O—CH₂—CH₂— CH₂—, W is O, and all of R¹, R², R³, R⁵ and R⁵* are all hydrogen. Such LNA nucleosides are disclosed in WO00/047599 and Morita et al, Bioorganic & Med. Chem. Lett. 12 73-76, which are hereby incorporated by reference, and include what are commonly known as 2′-O-4′C-ethylene bridged nucleic acids (ENA).

In some embodiments, the biradicle —X—Y— is —O—CH₂—, W is O, and all of R¹, R², R³, and one of R⁵ and R⁵* are hydrogen, and the other of R⁵ and R⁵* is other than hydrogen such as O₁₋₆ alkyl, such as methyl. Such 5′ substituted LNA nucleosides are disclosed in WO2007/134181 which is hereby incorporated by reference.

In some embodiments, the biradicle —X—Y— is —O—CR^{a}R^{b}—, wherein one or both of R^{a} and R^{b} are other than hydrogen, such as methyl, W is O, and all of R¹, R², R³, and one of R⁵ and R⁵* are hydrogen, and the other of R⁵ and R⁵* is other than hydrogen such as O₁₋₆ alkyl, such as methyl. Such bis modified LNA nucleosides are disclosed in WO2010/077578 which is hereby incorporated by reference.

In some embodiments, the biradicle —X—Y— designate the bivalent linker group —O—CH(CH₂OCH₃)—(2′ O-methoxyethyl bicyclic nucleic acid—Seth at al., 2010, J. Org. Chem. Vol 75(5) pp. 1569-81). In some embodiments, the biradicle —X—Y— designate the bivalent linker group —O—CH(CH₂CH₃)-(2′O-ethyl bicyclic nucleic acid—Seth at al., 2010, J. Org. Chem. Vol 75(5) pp. 1569-81). In some embodiments, the biradicle —X—Y— is —O—CHR^{a}—, W is O, and all of R¹, R², R³, R⁵ and R⁵* are all hydrogen. Such 6′ substituted LNA nucleosides are disclosed in WO10036698 and WO07090071 which are both hereby incorporated by reference.

In some embodiments, the biradicle —X—Y— is —O—CH(CH₂OCH₃)—, W is O, and all of R¹, R², R³, R⁵ and R⁵* are all hydrogen. Such LNA nucleosides are also known as cyclic MOEs in the art (cMOE) and are disclosed in WO07090071.

In some embodiments, the biradicle —X—Y— designate the bivalent linker group —O—CH(CH₃)—. —in either the R- or S-configuration. In some embodiments, the biradicle —X—Y— together designate the bivalent linker group —O—CH₂—O—CH₂— (Seth at al., 2010, J. Org. Chem). In some embodiments, the biradicle —X—Y— is —O—CH(CH₃)—, W is O, and all of R¹, R², R³, R⁵ and R⁵* are all hydrogen. Such 6′ methyl LNA nucleosides are also known as cET nucleosides in the art, and may be either (S)cET or (R)cET stereoisomers, as disclosed in WO07090071 (beta-D) and WO2010/036698 (alpha-L) which are both hereby incorporated by reference).

In some embodiments, the biradicle —X—Y— is —O—CR^{a}R^{b}—, wherein in neither R^{a} or R^{b} is hydrogen, W is O, and all of R¹, R², R³, R⁵ and R⁵* are all hydrogen. In some embodiments, R^{a} and R^{b} are both methyl. Such 6′ di-substituted LNA nucleosides are disclosed in WO 2009006478 which is hereby incorporated by reference.

In some embodiments, the biradicle —X—Y— is —S—CHR^{a}—, W is O, and all of R¹, R², R³, R⁵ and R⁵* are all hydrogen. Such 6′ substituted thio LNA nucleosides are disclosed in WO11156202 which is hereby incorporated by reference. In some 6′ substituted thio LNA embodiments R^{a} is methyl.

In some embodiments, the biradicle —X—Y— is —C(═CH2)—C(R^{a}R^{b})—, such as —C(═CH₂)—CH₂—, or —C(═CH₂)—CH(CH₃)—W is O, and all of R¹, R², R³, R⁵ and R⁵* are all hydrogen. Such vinyl carbo LNA nucleosides are disclosed in WO08154401 and WO09067647 which are both hereby incorporated by reference.

In some embodiments the biradicle —X—Y— is —N(—OR^{a})—, W is O, and all of R¹, R², R³, R⁵ and R⁵* are all hydrogen. In some embodiments R^{a} is C₁₋₆ alkyl such as methyl. Such LNA nucleosides are also known as N substituted LNAs and are disclosed in WO2008/150729 which is hereby incorporated by reference. In some embodiments, the biradicle —X—Y— together designate the bivalent linker group —O—NR^{a}—CH₃— (Seth at al., 2010, J. Org. Chem). In some embodiments the biradicle —X—Y— is —N(R^{a})—, W is O, and all of R¹, R², R³, R⁵ and R⁵* are all hydrogen. In some embodiments R^{a} is C₁₋₆ alkyl such as methyl.

In some embodiments, one or both of R⁵ and R⁵* is hydrogen and, when substituted the other of R⁵ and R⁵* is C₁₋₆ alkyl such as methyl. In such an embodiment, R¹, R², R³, may all be hydrogen, and the biradicle —X—Y— may be selected from —O—CH2— or —O—C(HCR^{a})—, such as —O—C(HCH3)—.

In some embodiments, the biradicle is —CR^{a}R^{b}—O—CR^{a}R^{b}—, such as CH₂—O—CH₂—, W is O and all of R¹, R², R³, R⁵ and R⁵* are all hydrogen. In some embodiments R^{a} is C₁₋₆ alkyl such as methyl. Such LNA nucleosides are also known as conformationally restricted nucleotides (CRNs) and are disclosed in WO2013036868 which is hereby incorporated by reference.

In some embodiments, the biradicle is —O—CR^{a}R^{b}—O—CR^{a}R^{b}—, such as O—CH₂—O—CH₂—, W is O and all of R¹, R², R³, R⁵ and R⁵* are all hydrogen. In some embodiments R^{a} is C₁₋₆ alkyl such as methyl. Such LNA nucleosides are also known as COC nucleotides and are disclosed in Mitsuoka et al., Nucleic Acids Research 2009 37(4), 1225-1238, which is hereby incorporated by reference.

It will be recognized than, unless specified, the LNA nucleosides may be in the beta-D or alpha-L stereoisoform.

Certain examples of LNA nucleosides are presented in Scheme 1.

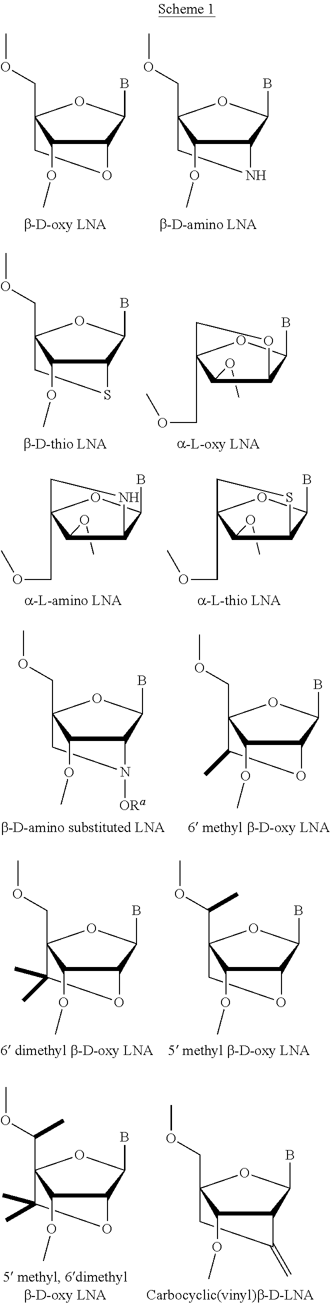

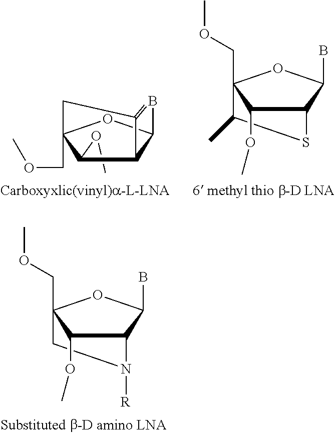

As illustrated in the examples, in some embodiments of the invention the LNA nucleosides in the oligonucleotides are beta-D-oxy-LNA nucleosides.

Nuclease Mediated Degradation

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the invention are capable of recruiting a nuclease, particularly and endonuclease, preferably endoribonuclease (RNase), such as RNase

H. Examples of oligonucleotide designs which operate via nuclease mediated mechanisms are oligonucleotides which typically comprise a region of at least 5 or 6 DNA nucleosides and are flanked on one side or both sides by affinity enhancing nucleosides, for example gapmers, headmers and tailmers.

RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference).

Gapmer

The term gapmer as used herein refers to an antisense oligonucleotide which comprises a region of RNase H recruiting oligonucleotides (gap) which is flanked 5′ and 3′ by regions which comprise one or more affinity enhancing modified nucleosides (flanks or wings). Various gapmer designs are described herein. Headmers and tailmers are oligonucleotides capable of recruiting RNase H where one of the flanks is missing, i.e. only one of the ends of the oligonucleotide comprises affinity enhancing modified nucleosides. For headmers the 3′ flank is missing (i.e. the 5′ flank comprises affinity enhancing modified nucleosides) and for tailmers the 5′ flank is missing (i.e. the 3′ flank comprises affinity enhancing modified nucleosides).

LNA Gapmer

The term LNA gapmer is a gapmer oligonucleotide wherein at least one of the affinity enhancing modified nucleosides is an LNA nucleoside.

Mixed Wing Gapmer

The term mixed wing gapmer or mixed flank gapmer refers to a LNA gapmer wherein at least one of the flank regions comprise at least one LNA nucleoside and at least one non-LNA modified nucleoside, such as at least one 2′ substituted modified nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA and 2′-F-ANA nucleoside(s). In some embodiments the mixed wing gapmer has one flank which comprises only LNA nucleosides (e.g. 5′ or 3′) and the other flank (3′ or 5′ respectfully) comprises 2′ substituted modified nucleoside(s) and optionally LNA nucleosides.

Gapbreaker

The term “gapbreaker oligonucleotide” is used in relation to a gapmer capable of maintaining RNAseH recruitment even though the gap region is disrupted by a non-RNaseH recruiting nucleoside (a gap-breaker nucleoside, E) such that the gap region comprise less than 5 consecutive DNA nucleosides. Non-RNaseH recruiting nucleosides are for example nucleosides in the 3′ endo conformation, such as LNA's where the bridge between C2′ and C4′ of the ribose sugar ring of a nucleoside is in the beta conformation, such as beta-D-oxy LNA or ScET nucleoside. The ability of gapbreaker oligonucleotide to recruit RNaseH is typically sequence or even compound specific—see Rukov et al. 2015 Nucl. Acids Res. Vol. 43 pp. 8476-8487, which discloses “gapbreaker” oligonucleotides which recruit RNaseH which in some instances provide a more specific cleavage of the target RNA.

In some embodiments, the oligonucleotide of the invention is a gapbreaker oligonucleotide. In some embodiments the gapbreaker oligonucleotide comprise a 5′-flank (F), a gap (G) and a 3′-flank (F′), wherein the gap is disrupted by a non-RNaseH recruiting nucleoside (a gap-breaker nucleoside, E) such that the gap contain at least 3 or 4 consecutive DNA nucleosides. In some embodiments the gapbreaker nucleoside (E) is an LNA nucleoside where the bridge between C2′ and C4′ of the ribose sugar ring of a nucleoside is in the beta conformation and is placed within the gap region such that the gap-breaker LNA nucleoside is flanked 5′ and 3′ by at least 3 (5′) and 3 (3′) or at least 3 (5′) and 4 (3′) or at least 4(5′) and 3(3′) DNA nucleosides, and wherein the oligonucleotide is capable of recruiting RNaseH.

The gapbreaker oligonucleotide can be represented by the following formulae:

F-G-E-G-F′; in particular F₁₋₇-G₃₋₄-E₁-G₃₋₄-F₁₋₇

D′-F-G-F′, in particular D′₁₋₃-F₁₋₇- G₃₋₄-E₁-G₃₋₄-F₁₋₇

F-G-F′-D″, in particular F₁₋₇ G₃-₄⁻E₁-G₃₋₄F₁₋₇D″₁₋₃

D′-F-G-F′-D″, in particular D′₁₋₃-F₁₋₇- G₃₋₄-E₁-G₃₋₄-F₁₋₇-D″₁₋₃

Where region D′ and D″ are as described in the section “Gapmer design”.

In some embodiments the gapbreaker nucleoside (E) is a beta-D-oxy LNA or ScET or another beta-LNA nucleosides shown in Scheme 1).

Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. A the same time the conjugate may serve to reduce activity of the oligonucleotide in non-target cell types, tissues or organs, e.g. off target activity or activity in non-target cell types, tissues or organs. WO 93/07883 and WO2013/033230 provides suitable conjugate moieties, which are hereby incorporated by reference. Further suitable conjugate moieties are those capable of binding to the asialoglycoprotein receptor (ASGPr). In particular tri-valent N-acetylgalactosamine conjugate moieties are suitable for binding to the ASGPr, see for example WO 2014/076196, WO 2014/207232 and WO 2014/179620 (hereby incorporated by reference).

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers are described in more detail in WO 2014/076195 (hereby incorporated by reference).

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In some embodiments the linker (region Y) is a C6 amino alkyl group.

Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

### DETAILED DESCRIPTION OF THE INVENTION

The Oligonucleotides of the Invention

The invention relates to oligonucleotides capable of inhibiting the expression of NF-κB2. The modulation is may achieved by hybridizing to a target nucleic acid encoding NF-κB2 or which is involved in the regulation of NF-κB2. The target nucleic acid may be a mammalian NFKB2 sequence, such as SEQ ID NO 21.

In some embodiments the antisense oligonucleotide of the invention is capable of modulating the expression of the target by inhibiting or down-regulating it. Preferably, such modulation produces an inhibition of expression of at least 20% compared to the normal expression level of the target, more preferably at least 30%, 40%, 50%, 60%, 70%, 80%, or 90% inhibition compared to the normal expression level of the target. In some embodiments oligonucleotides of the invention may be capable of inhibiting expression levels of NFKB2 mRNA by at least 60% or 70% in vitro using HEK-293 or HeLa cells. In some embodiments compounds of the invention may be capable of inhibiting expression levels of NF-κB2 protein by at least 50% in vitro using HEK-293 or HeLa cells. Suitably, the examples provide assays which may be used to measure NFKB2 RNA or protein inhibition. The target modulation is triggered by the hybridization between a contiguous nucleotide sequence of the oligonucleotide and the target nucleic acid. In some embodiments the oligonucleotide of the invention comprises mismatches between the oligonucleotide and the target nucleic acid. Despite mismatches hybridization to the target nucleic acid may still be sufficient to show a desired modulation of NF-κB2 expression. Reduced binding affinity resulting from mismatches may advantageously be compensated by increased number of nucleotides in the oligonucleotide and/or an increased number of modified nucleosides capable of increasing the binding affinity to the target, such as 2′ modified nucleosides, including LNA, present within the oligonucleotide sequence.

An aspect of the present invention relates to an antisense oligonucleotide which consists or comprises a contiguous nucleotide sequence of 10 to 30 nucleotides in length with at least 90% complementarity to a human NFKB2 sequence.

In some embodiments, the oligonucleotide comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid.

In some embodiments the oligonucleotide of the invention, or contiguous nucleotide sequence thereof is fully complementary (100% complementary) to a region of the target nucleic acid, or in some embodiments may comprise one or two mismatches between the oligonucleotide and the target nucleic acid.

In some embodiments the oligonucleotide comprises a contiguous nucleotide sequence of 10 to 30 nucleotides in length with at least 90% complementary, such as fully (or 100%) complementary, to a region of a sequence selected from SEQ ID NOs 11, 12, 13, 14, 15, 16, 17, 18, 19, 20, and 22.

In some embodiments, the oligonucleotide of the invention comprises or consists of 8 to 35 nucleotides in length, such as from 10 to 30, such as 11 to 22, such as from 12 to 18, such as from 13 to 17 or 14 to 16 contiguous nucleotides in length. In some embodiments the oligonucleotide comprises or consists of 13, 14, 15, 16 or 17 nucleotides in length.

In some embodiments, the oligonucleotide or contiguous nucleotide sequence thereof comprises or consists of 22 or less nucleotides, such as 20 or less nucleotides, such as 18 or less nucleotides, such as 14, 15, 16 or 17 nucleotides. It is to be understood that any range given herein includes the range endpoints. Accordingly, if an oligonucleotide is said to include from 10 to 30 nucleotides, both 10 and 30 nucleotides are included.

In some embodiments, the contiguous nucleotide sequence comprises or consists of 8, 9, 10, 11, 12, 13, 14, 15, 16, 17, 18, 19, 20, 21, 22, 23, 24, 25, 26, 27, 28, 29 or 30 contiguous nucleotides in length. In some embodiments, the oligonucleotide comprises or consists of 14, 15 or 16 nucleotides in length.

In some embodiments, the oligonucleotide or contiguous nucleotide sequence comprises or consists of a sequence selected from the group consisting SEQ ID NO 1, 2, 3, 4, 5, 6, 7, 8, 9 and 10, or at least 12 contiguous nucleotides thereof.

In some embodiments, the oligonucleotide or contiguous nucleotide sequence comprises or consists of a sequence selected from the group consisting SEQ ID NO 1, 2, 3, 4, 6, 7, 8, 9 and 10, or at least 12 contiguous nucleotides thereof.

In some embodiments, the oligonucleotide or contiguous nucleotide sequence comprises or consists of a sequence selected from the group consisting SEQ ID NO 1, 2, 3, 4, 5, 6, 7, 8, 9 and 10, or at least 14 contiguous nucleotides thereof.

In some embodiments, the oligonucleotide or contiguous nucleotide sequence comprises or consists of a sequence selected from the group consisting SEQ ID NO 1, 2, 3, 4, 5, 6, 7, 8, 9 and 10, or at least 15 contiguous nucleotides thereof.

In some embodiments, the oligonucleotide or contiguous nucleotide sequence comprises or consists of a sequence selected from the group consisting SEQ ID NO 5 or at least 13, 14, 15 or 16 contiguous nucleotides thereof.

Oligonucleotide Design

Oligonucleotide design refers to the pattern of nucleoside sugar modifications in the oligonucleotide sequence. The oligonucleotides of the invention comprise sugar-modified nucleosides and may also comprise DNA or RNA nucleosides. In some embodiments, the oligonucleotide comprises sugar-modified nucleosides and DNA nucleosides. Incorporation of modified nucleosides into the oligonucleotide of the invention may enhance the affinity of the oligonucleotide for the target nucleic acid. In that case, the modified nucleosides can be referred to as affinity enhancing modified nucleotides, the modified nucleosides may also be termed units.

In an embodiment, the oligonucleotide comprises at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides. In an embodiment the oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides.

In an embodiment, the oligonucleotide comprises one or more sugar modified nucleosides, such as 2′ sugar modified nucleosides. Preferably the oligonucleotide of the invention comprise the one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. Even more preferably the one or more modified nucleoside is a locked nucleic acid (LNA).

In a further embodiment the oligonucleotide comprises at least one modified internucleoside linkage. In some embodiments all the internucleoside linkages within the contiguous nucleotide sequence are phosphorothioate or boranophosphate internucleoside linkages. In some embodiments all the internucleotide linkages in the contiguous sequence of the oligonucleotide are phosphorothioate linkages.

In some embodiments, the oligonucleotide of the invention comprises at least one LNA nucleoside, such as 1, 2, 3, 4, 5, 6, 7, or 8 LNA nucleosides, such as from 2 to 6 LNA nucleosides, such as from 3 to 7 LNA nucleosides, 4 to 8 LNA nucleosides or 3, 4, 5, 6, 7 or 8 LNA nucleosides. In some embodiments, at least 75% of the modified nucleosides in the oligonucleotide are LNA nucleosides, such as 80%, such as 85%, such as 90% of the modified nucleosides are LNA nucleosides. In a still further embodiment all the modified nucleosides in the oligonucleotide are LNA nucleosides. In a further embodiment, the oligonucleotide may comprise both beta-D-oxy-LNA, and one or more of the following LNA nucleosides: thio-LNA, amino-LNA, oxy-LNA, and/or ENA in either the beta-D or alpha-L configurations or combinations thereof. In a further embodiment, all LNA cytosine units are 5-methyl-cytosine. In some embodiments the oligonucleotide or contiguous nucleotide sequence has at least 1 LNA nucleoside at the 5′ end and at least 2 LNA nucleosides at the 3′ end of the nucleotide sequence.

In some embodiments, the oligonucleotide of the invention comprises at least one modified nucleoside which is a 2′-MOE-RNA nucleoside, such as 2, 3, 4, 5, 6, 7, 8, 9 or 10 2′-MOE-RNA nucleosides. In some embodiments, at least one of said modified nucleoside is 2′-fluoro DNA, such as 2, 3, 4, 5, 6, 7, 8, 9 or 10 2′-fluoro-DNA nucleosides.

In some embodiments, the oligonucleotide of the invention comprises at least one LNA nucleoside and at least one 2′ substituted modified nucleoside.

In some embodiments of the invention, the oligonucleotide comprise both 2′ sugar modified nucleosides and DNA units. Preferably the oligonucleotide comprises both LNA and DNA nucleosides (units). Preferably, the combined total of LNA and DNA units is 8-30, such as 10 -25, preferably 12-22, such as 12-18, even more preferably 11-16. In some embodiments of the invention, the nucleotide sequence of the oligonucleotide, such as the contiguous nucleotide sequence consists of at least one or two LNA nucleosides and the remaining nucleosides are DNA units. In some embodiments the oligonucleotide comprises only LNA nucleosides and naturally occurring nucleosides (such as RNA or DNA, most preferably DNA nucleosides), optionally with modified internucleoside linkages such as phosphorothioate.

In an embodiment of the invention the oligonucleotide of the invention is capable of recruiting RNase H.

The structural design of the oligonucleotide of the invention may be selected from gapmers, gapbreakers, headmers and tailmers. In some embodiments the oligonucleotide of the invention is a gapmer.

Gapmer Design

In some embodiments the oligonucleotide of the invention has a gapmer design or structure also referred herein merely as “Gapmer”. In a gapmer structure the oligonucleotide comprises at least three distinct structural regions a 5′-flank, a gap and a 3′-flank, F-G-F′ in ‘5→3’ orientation. In this design, flanking regions F and F′ (also termed wing regions) comprise a contiguous stretch of modified nucleosides, which are complementary to the NFKB2 target nucleic acid, while the gap region, G, comprises a contiguous stretch of nucleotides which are capable of recruiting a nuclease, preferably an endonuclease such as RNase, for example RNase H, when the oligonucleotide is in duplex with the target nucleic acid. Nucleosides which are capable of recruiting a nuclease, in particular RNase H, can be selected from the group consisting of DNA, alpha-L-oxy-LNA, 2′-Flouro-ANA and UNA. Regions F and F′, flanking the 5′ and 3′ ends of region G, preferably comprise non-nuclease recruiting nucleosides (nucleosides with a 3′ endo structure), more preferably one or more affinity enhancing modified nucleosides. In some embodiments, the 3′ flank comprises at least one LNA nucleoside, preferably at least 2 LNA nucleosides. In some embodiments, the 5′ flank comprises at least one LNA nucleoside. In some embodiments both the 5′ and 3′ flanking regions comprise a LNA nucleoside. In some embodiments all the nucleosides in the flanking regions are LNA nucleosides. In other embodiments, the flanking regions may comprise both LNA nucleosides and other nucleosides (mixed flanks), such as DNA nucleosides and/or non-LNA modified nucleosides, such as 2′ substituted nucleosides. In this case the gap is defined as a contiguous sequence of at least 5 RNase H recruiting nucleosides (nucleosides with a 2′ endo structure, preferably DNA) flanked at the 5′ and 3′ end by an affinity enhancing modified nucleoside, preferably LNA, such as beta-D-oxy-LNA. Consequently, the nucleosides of the 5′ flanking region and the 3′ flanking region which are adjacent to the gap region are modified nucleosides, preferably non-nuclease recruiting nucleosides.

Region F

Region F (5′ flank or 5′ wing) attached to the ‘5 end of region G comprises, contains or consists of at least one modified nucleoside such as at least 2, at least 3, at least 4, at least 5, at least 6, at least 7 modified nucleosides. In an embodiment region F comprises or consists of from 1 to 7 modified nucleosides, such as from 2 to 6 modified nucleosides, such as from 2 to 5 modified nucleosides, such as from 2 to 4 modified nucleosides, such as from 1 to 3 modified nucleosides, such as 1, 2, 3 or 4 modified nucleosides. The F region is defined by having at least on modified nucleoside at the 5’ end and at the 3′ end of the region.

In some embodiments, the modified nucleosides in region F have a 3′ endo structure.

In an embodiment, one or more of the modified nucleosides in region F are 2′ modified nucleosides. In one embodiment all the nucleosides in Region F are 2′ modified nucleosides.

In another embodiment region F comprises DNA and/or RNA in addition to the 2′ modified nucleosides. Flanks comprising DNA and/or RNA are characterized by having a 2′ modified nucleoside in the 5′ end and the 3′end (adjacent to the G region) of the F region. In one embodiment the region F comprise DNA nucleosides, such as from 1 to 3 contiguous DNA nucleosides, such as 1 to 3 or 1 to 2 contiguous DNA nucleosides. The DNA nucleosides in the flanks should preferably not be able to recruit RNase H. In some embodiments the 2′ modified nucleosides and DNA and/or RNA nucleosides in the F region alternate with 1 to 3 2′ modified nucleosides and 1 to 3 DNA and/or RNA nucleosides. Such flanks can also be termed alternating flanks. The length of the 5′ flank (region F) in oligonucleotides with alternating flanks may be 4 to 10 nucleosides, such as 4 to 8, such as 4 to 6 nucleosides, such as 4, 5, 6 or 7 modified nucleosides. In some embodiments only the 5′ flank of the oligonucleotide is alternating. Specific examples of region F with alternating nucleosides are

2′₁₋₃-N′₁₋₄-2′₁₋₃

2′₁₋₂-N′₁₋₂-2′₁₋₂-N′₁₋₂-2′₁₋₂

Where 2′ indicates a modified nucleoside and N′ is a RNA or DNA. In some embodiments all the modified nucleosides in the alternating flanks are LNA and the N′ is DNA.In a further embodiment one or more of the 2′ modified nucleosides in region F are selected from 2′-)-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments the F region comprises both LNA and a 2′ substituted modified nucleoside. These are often termed mixed wing or mixed flank oligonucleotides.

In one embodiment of the invention all the modified nucleosides in region F are LNA nucleosides. In a further embodiment all the nucleosides in Region F are LNA nucleosides. In a further embodiment the LNA nucleosides in region F are independently selected from the group consisting of oxy-LNA, thio-LNA, amino-LNA, cET, and/or ENA, in either the beta-D or alpha-L configurations or combinations thereof. In some embodiments region F comprise at least 1 beta-D-oxy LNA unit, at the 5′ end of the contiguous sequence.

Region G

Region G (gap region) preferably comprise, contain or consist of at least 4, such as at least 5, such as at least 6, at least 7, at least 8, at least 9, at least 10, at least 11, at least 12, at least 13, at least 14, at least 15 or at least 16 consecutive nucleosides capable of recruiting the aforementioned nuclease, in particular RNaseH. In a further embodiment region G comprise, contain or consist of from 5 to 12, or from 6 to 10 or from 7 to 9, such as 8 consecutive nucleotide units capable of recruiting aforementioned nuclease.

The nucleoside units in region G, which are capable of recruiting nuclease are in an embodiment selected from the group consisting of DNA, alpha-L-LNA, C4′ alkylated DNA (as described in PCT/EP2009/050349 and Vester et al., Bioorg. Med. Chem. Lett. 18 (2008) 2296 -2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue.

In a still further embodiment at least one nucleoside unit in region G is a DNA nucleoside unit, such as from 1 to 12 DNA units, such as 2, 3, 4, 5, 6, 7, 8, 9, 10 or 11 DNA units, preferably from 2 to 12 DNA units, such as from 4 to 12 DNA units, more preferably from 5 to 11, or from 2 to 10, 4 to 10 or 6 to 10 DNA units, such as from 7 to 10 DNA units, such as 8, 9 or 10 DNA units. In some embodiments, region G consists of 100% DNA units. In some embodiment G consists of from 8-12 DNA units.

In further embodiments the region G may consist of a mixture of DNA and other nucleosides capable of mediating RNase H cleavage. Region G may consist of at least 50% DNA, more preferably 60%, 70% or 80% DNA, and even more preferred 90% or 95% DNA.

In a still further embodiment at least one nucleoside unit in region G is an alpha-L-LNA nucleoside unit, such as at least one alpha-L-LNA, such as 2, 3, 4, 5, 6, 7, 8 or 9 alpha-L-LNA. In a further embodiment, region G comprises the least one alpha-L-LNA is alpha-L-oxy-LNA. In a further embodiment region G comprises a combination of DNA and alpha-L-LNA nucleoside units.

In some embodiments the size of the contiguous sequence in region G may be longer, such as 12, 13, 14, 15, 16, 17, 18, 19 or 20 nucleoside units.

In some embodiments, nucleosides in region G have a 2′ endo structure.

In some embodiments region G may comprise a gapbreaker nucleoside, leading to a gapbreaker oligonucleotide, which is capable of recruiting RNase H.

Region F′

Region F′ (3′ flank or 3′ wing) attached to the ‘3 end of region G comprises, contains or consists of at least one modified nucleoside such as at least 2, at least 3, at least 4, at least 5, at least 6, at least 7 modified nucleosides. In an embodiment region F’ comprise or consist of from 1 to 7 modified nucleosides, such as from 2 to 6 modified nucleoside, such as from 2 to 4 modified nucleosides, such as from 1 to 3 modified nucleosides, such as 1, 2, 3 or 4 modified nucleosides. The F′ region is defined by having at least on modified nucleoside at the 5′ end and at the 3′ end of the region.

In some embodiments, the modified nucleosides in region F′ have a 3′ endo structure.

In an embodiment, one or more of the modified nucleosides in region F′ are 2′ modified nucleosides. In one embodiment all the nucleosides in Region F′ are 2′ modified nucleosides.

In an embodiment, one or more of the modified nucleosides in region F′ are 2′ modified nucleosides.

In one embodiment all the nucleosides in Region F′ are 2′ modified nucleosides. In another embodiment region F′ comprises DNA or RNA in addition to the 2′ modified nucleosides. Flanks comprising DNA or RNA are characterized by having a 2′ modified nucleoside in the 5′ end (adjacent to the G region) and the 3′end of the F′ region. In one embodiment the region F′ comprises DNA nucleosides, such as from 1 to 4 contiguous DNA nucleosides, such as 1 to 3 or 1 to 2 contiguous DNA nucleosides. The DNA nucleosides in the flanks should preferably not be able to recruit RNase H. In some embodiments the 2′ modified nucleosides and DNA and/or RNA nucleosides in the F′ region alternate with 1 to 3 2′ modified nucleosides and 1 to 3 DNA and/or RNA nucleosides, such flanks can also be termed alternating flanks. The length of the 3′ flank (region F′) in oligonucleotides with alternating flanks may be 4 to 10 nucleosides, such as 4 to 8, such as 4 to 6 nucleosides, such as 4, 5, 6 or 7 modified nucleosides. In some embodiments only the 3′ flank of the oligonucleotide is alternating. Specific examples of region F′ with alternating nucleosides are

2′₁₋₂-N′₁₋₄-2′₁₋₄

2′₁₋₂-N′₁₋₂-2′₁₋₂-N′₁₋₂-2′₁₋₂

Where 2′ indicates a modified nucleoside and N′ is a RNA or DNA. In some embodiments all the modified nucleosides in the alternating flanks are LNA and the N′ is DNA. In a further embodiment modified nucleosides in region F′ are selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments the F′ region comprises both LNA and a 2′ substituted modified nucleoside. These are often termed mixed wing or mixed flank oligonucleotides.

In one embodiment of the invention all the modified nucleosides in region F′ are LNA nucleosides. In a further embodiment all the nucleosides in Region F′ are LNA nucleosides. In a further embodiment the LNA nucleosides in region F′ are independently selected from the group consisting of oxy-LNA, thio-LNA, amino-LNA, cET and/or ENA, in either the beta-D or alpha-L configurations or combinations thereof. In some embodiments region F′ has at least 2 beta-D-oxy LNA unit, at the 3′ end of the contiguous sequence.

Region D′ and D″

Region D′ and D″ can be attached to the 5′ end of region F or the 3′ end of region F′, respectively.

Region D′ or D″ may independently comprise 1, 2, 3, 4 or 5 additional nucleotides, which may be complementary or non-complementary to the target nucleic acid. In this respect the oligonucleotide of the invention, may in some embodiments comprise a contiguous nucleotide sequence capable of modulating the target which is flanked at the 5′ and/or 3′ end by additional nucleotides. Such additional nucleotides may serve as a nuclease susceptible biocleavable linker (see definition of linkers). In some embodiments the additional 5′ and/or 3′ end nucleotides are linked with phosphodiester linkages, and may be DNA or RNA. In another embodiment, the additional 5′ and/or 3′ end nucleotides are modified nucleotides which may for example be included to enhance nuclease stability or for ease of synthesis. In an embodiment of the oligonucleotide, the invention comprises a region D′ and/or D″ in addition to the contiguous nucleotide sequence.

In some embodiments the oligonucleotide of the invention may consist of the contiguous nucleotide sequence and region D′ and/or D″, and a conjugation group covalently attached to region D′ or D″.

The gapmer oligonucleotide of the present invention can be represented by the following formulae:

F-G-F′; in particular F₁₋₇-G₄₋₁₂-F′₁₋₇

D′-F-G-F′, in particular D′₁₋₃-F₁₋₇-G₄₋₁₂-F₁₋₇

F-G-F′-D″, in particular F₁₋₇-G₄₋₁₂-F′₁₋₇-D″₁₋₃

D′-F-G-F′-D″, in particular D′₁₋₃-F₁₋₇-G₄₋₁₂-F₁₋₇-D″₁₋₃

The preferred number and types of nucleosides in regions F, G and F′, D′ and D″ have been described above.

The oligonucleotide conjugates of the present invention have a region C covalently attached to either the 5′ or 3′ end of the oligonucleotide, in particular the gapmer oligonucleotides presented above.

In one embodiment the oligonucleotide conjugate of the invention comprises a oligonucleotide with the formula 5′-D′-F-G-F′-3′ or 5′-F-G-F′-D″-3′, where region F and F′ independently comprise 1-7 modified nucleosides, G is a region between 6 and 16 nucleosides which are capable of recruiting RNaseH and region D′ or D″ comprise 1-5 phosphodiester linked nucleosides. Preferably region D′ or D″ is present in the end of the oligonucleotide where conjugation to a conjugate moiety is contemplated.

Examples of oligonucleotides with alternating flanks can be represented by the following formulae:

2′₁₋₃-N′₁₋₄-2′₁₋₃-G₆₋₁₂-2′₁₋₂-N′₁₋₄-2′₁₋₄

2′₁₋₂-N′₁₋₂-2′₁₋₂-N₁₋₂-2′₁₋₂-G₆₋₁₂-2′₁₋₂-N₁₋₂-2′₁₋₂-N′₁₋₂-2′₁₋₂

F-G₆₋₁₂-2′₁₋₂-N′₁₋₄-2′₁₋₄

F-G₆₋₁₂-2′₁₋₂-N₁₋₂-2′₁₋₂-N₁₋₂-240 ₁₋₂

2′₁₋₃-N′₁₋₄-2′₁₋₃-G₆₋₁₂-F

2′₁₋₂-N′₁₋₂-2′₁₋₂-N₁₋₂-2′₁₋₂-G₆₋₁₂-F

Where a flank is indicated by F or F′ it only contains 2′ modified nucleosides, such as LNA nucleosides. The preferred number and types of nucleosides in the alternating regions, and region F, G and F′, D′ and D″ have been described above.

In some embodiments the oligonucleotide is a gapmer consisting of 10, 11, 12, 13, 14, 15 or 16 nucleotides in length, wherein each of regions F and F′ independently consists of 1, 2, 3 or 4 modified nucleoside units complementary to the NFKB2 target nucleic acid and region G consists of 7, 8, 9, or 10 nucleoside units, capable of recruiting nuclease when in duplex with the NFKB2 target nucleic acid.

In a further embodiments, the oligonucleotide is a gapmer wherein each of regions F and F′ independently consists of 3, 4, 5 or 6 modified nucleoside units, such as nucleoside units containing a 2′-O-methoxyethyl-ribose sugar (2′-MOE) or nucleoside units containing a 2′-fluoro-deoxyribose sugar and/or LNA units, and region G consists of 8, 9, 10, 11 or 12 nucleoside units, such as DNA units or other nuclease recruiting nucleosides such as alpha-L-LNA or a mixture of DNA and nuclease recruiting nucleosides.

In a further specific embodiment, the oligonucleotide is a gapmer wherein each of regions F and F′ region consists of two LNA units each, and region G consists of 8, 9 or 10 nucleoside units, preferably DNA units. Specific gapmer designs of this nature include 2-8-2, 2-9-2 and 2-10-2.

In a further specific embodiment, the oligonucleotide is a gapmer wherein each of regions F and F′ independently consists of three LNA units, and region G consists of 8, 9 or 10 nucleoside units, preferably DNA units. Specific gapmer designs of this nature include 3-8-3, 3-9-3 and 3-10-3.

In a further specific embodiment, the oligonucleotide is a gapmer wherein each of regions F and F′ consists of four LNA units each, and region G consists of 8 or 9 or 10 nucleoside units, preferably DNA units. Specific gapmer designs of this nature include 4-8-4, 4-9-4 and 4-10-4

Specific gapmer designs of this nature include F-G-F′ designs selected from a group consisting of a gap with 6 nucleosides and independently 1 to 4 modified nucleosides in the wings including 1-6-1, 1-6-2, 2-6-1, 1-6-3, 3-6-1, 1-6-4, 4-6-1, 2-6-2, 2-6-3, 3-6-2 2-6-4, 4-6-2, 3-6-3, 3-6-4 and 4-6-3 gapmers.

Specific gapmer designs of this nature include F-G-F′ designs selected from a group consisting of a gap with 7 nucleosides and independently 1 to 4 modified nucleosides in the wings including 1-7-1, 2-7-1, 1-7-2, 1-7-3, 3-7-1, 1-7-4, 4-7-1, 2-7-2, 2-7-3, 3-7-2, 2-7-4, 4-7-2, 3-7-3, 3-7-4, 4-7-3 and 4-7-4 gapmers.

Specific gapmer designs of this nature include F-G-F′ designs selected from a group consisting of a gap with 8 nucleosides and independently 1 to 4 modified nucleosides in the wings including 1-8-1, 1-8-2, 1-8-3, 3-8-1, 1-8-4, 4-8-1,2-8-1, 2-8-2, 2-8-3, 3-8-2, 2-8-4, 4-8-2, 3-8-3, 3-8-4, 4-8-3, and 4-8-4 gapmers.

Specific gapmer designs of this nature include F-G-F′ designs selected from a group consisting of a gap with 9 nucleosides and independently 1 to 4 modified nucleosides in the wings including, 1-9-1, 2-9-1, 1-9-2, 1-9-3, 3-9-1, 1-9-4, 4-9-1, 2-9-2, 2-9-3, 3-9-2, 2-9-4, 4- 9-2, 3-9-3, 3-9-4, 4-9-3 and 4-9-4 gapmers.

Specific gapmer designs of this nature include F-G-F′ designs selected from a group consisting of a gap with 10 nucleosides including, 1-10-1, 2-10-1, 1-10-2, 1-10-3, 3-10-1, 1-10-4, 4-10-1, 2-10-2, 2-10-3, 3-10-2, 2-10-4, 4-10-2, 3-10-3, 3-10-4, 4-10-3 and 4-10-4 gapmers.

In some embodiments the F-G-F′ design is selected from 3-11-2, 2-10-3, 4-9-2, 2-10-4, 4-10-2, 3-10-3, 4-10-2, 3-9-3, 4-9-2, and 3-10-3.

In some embodiments, the F-G-F′ design may, optionally, further include region D′ and/or D″, which may have 1, 2 or 3 nucleoside units, such as DNA units. In some embodiments, the nucleosides in region F and F′ are modified nucleosides, while nucleotides in region G are preferably unmodified nucleosides, such as DNA nucleosides.

In each design, in some embodiments the modified nucleoside is LNA.

In another embodiment all the internucleoside linkages in the gap in a gapmer are phosphorothioate and/or boranophosphate linkages. In another embodiment all the internucleoside linkages in the flanks (F and F′ region) in a gapmer are phosphorothioate and/or boranophosphate linkages. In another preferred embodiment all the internucleoside linkages in the D′ and D″ region in a gapmer are phosphodiester linkages.

For specific gapmers as disclosed herein, when the cytosine (C) residues are annotated as 5-methyl-cytosine, in various embodiments, one or more of the Cs present in the oligonucleotide may be unmodified C residues.

In a particular embodiment, the gapmer is a so-called shortmer as described in WO2008/113832 incorporated herein by reference.

Further gapmer designs are disclosed in WO2004/046160, WO2007/146511 and incorporated by reference.

For certain embodiments of the invention, the oligonucleotide is selected from the group of oligonucleotide compounds with CMP-ID-NO: 1,1; 2,1; 3,1; 4,1; 5,1; 6,1; 7,1; 8,1; 9,1; and 10,1.

Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand). In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution. The invention provides a sodium or potassium salt of the oligonucleotide of the invention.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

Oligonucleotides or oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration.

The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the oligonucleotide or oligonucleotide conjugate of the invention is a prodrug. In particular with respect to oligonucleotide conjugates the conjugate moiety is cleaved of the oligonucleotide once the prodrug is delivered to the site of action, e.g. the target cell.

Applications

The oligonucleotides of the invention may be utilized as research reagents for, for example, diagnostics, therapeutics and prophylaxis.

In research, such oligonucleotides may be used to specifically modulate the synthesis of NF-κB2 protein in cells (e.g. in vitro cell cultures) and experimental animals thereby facilitating functional analysis of the target or an appraisal of its usefulness as a target for therapeutic intervention. Typically the target modulation is achieved by degrading or inhibiting the mRNA producing the protein, thereby prevent protein formation or by degrading or inhibiting a modulator of the gene or mRNA producing the protein.

If employing the oligonucleotide of the invention in research or diagnostics the target nucleic acid may be a cDNA or a synthetic nucleic acid derived from DNA or RNA.

The present invention provides an in vivo or in vitro method for modulating NF-κB2 expression in a target cell which is expressing NF-κB2, said method comprising administering an oligonucleotide of the invention in an effective amount to said cell.

In some embodiments, the target cell, is a mammalian cell in particular a human cell. The target cell may be an in vitro cell culture or an in vivo cell forming part of a tissue in a mammal.

In diagnostics the oligonucleotides may be used to detect and quantitate NFKB2 expression in cell and tissues by northern blotting, in-situ hybridisation or similar techniques.

For therapeutics, an animal or a human, suspected of having a disease or disorder, which can be treated by modulating the expression of NF-κB2, such as cancer, inflammation or an inflammatory disease, or an autoimmune disease.

In some embodiments, the invention relates to oligonucleotides, oligonucleotide conjugates or pharmaceutical compositions for use in the treatment of diseases or disorders selected from the group consisting of rheumatoid arthritis, ulcerative colitis or B cell lymphomas

The invention provides methods for treating or preventing a disease, comprising administering a therapeutically or prophylactically effective amount of an oligonucleotide, an oligonucleotide conjugate or a pharmaceutical composition of the invention to a subject suffering from or susceptible to the disease.

The invention also relates to an oligonucleotide, a composition or a conjugate as defined herein for use as a medicament.

The oligonucleotide, oligonucleotide conjugate or a pharmaceutical composition according to the invention is typically administered in an effective amount.

The invention also provides for the use of the oligonucleotide or oligonucleotide conjugate of the invention as described for the manufacture of a medicament for the treatment of a disorder as referred to herein, or for a method of the treatment of as a disorder as referred to herein.

The disease or disorder, as referred to herein, is associated with expression of NF-κB2. In some embodiments disease or disorder may be associated with a mutation in the NFKB2 gene or a gene whose protein product is associated with or interacts with NF-κB2. Therefore, in some embodiments, the target nucleic acid is a mutated form of the NFKB2 sequence and in other embodiments, the target nucleic acid is a regulator of the NFKB2 sequence.

The methods of the invention are preferably employed for treatment or prophylaxis against diseases caused by abnormal levels and/or activity of NF-κB2.

The invention further relates to use of an oligonucleotide, oligonucleotide conjugate or a pharmaceutical composition as defined herein for the manufacture of a medicament for the treatment of abnormal levels and/or activity of NF-κB2.

In some embodiments, the invention relates to oligonucleotides, oligonucleotide conjugates or pharmaceutical compositions for use in the treatment of diseases or disorders selected from the group consisting of cancer, inflammation and inflammatory disorders, and autoimmune diseases.

In some embodiments, the invention relates to oligonucleotides, oligonucleotide conjugates or pharmaceutical compositions for use in the treatment of diseases or disorders selected from the group consisting of multiple sclerosis, Crohn's disease and rheumatoid arthritis.

In some embodiments, the invention relates to oligonucleotides, oligonucleotide conjugates or pharmaceutical compositions for use in the reducing inflammation in a patient who is in need to reduced inflammation.

In some embodiments, the invention relates to oligonucleotides, oligonucleotide conjugates or pharmaceutical compositions for use in the reducing cytokine levels in a patient who is in need to reduced cytokines.

In some embodiments, the invention relates to oligonucleotides, oligonucleotide conjugates or pharmaceutical compositions for use in the treatment of septic shock.

Administration

The oligonucleotides or pharmaceutical compositions of the present invention may be administered by any suitable means, such as via parenteral administration (such as, intravenous, subcutaneous, or intra-muscular.

In some embodiments the active oligonucleotide or oligonucleotide conjugate is administered intravenously. In another embodiment the active oligonucleotide or oligonucleotide conjugate is administered subcutaneously.

In some embodiments, the oligonucleotide, oligonucleotide conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

The invention also provides for the use of the oligonucleotide or oligonucleotide conjugate of the invention as described for the manufacture of a medicament wherein the medicament is in a dosage form for subcutaneous administration.

Combination Therapies

In some embodiments the oligonucleotide, oligonucleotide conjugate or pharmaceutical composition of the invention is for use in a combination treatment with another therapeutic agent.

### EMBODIMENTS

1. An LNA antisense oligonucleotide of 10 to 30 contiguous nucleotides in length, targeting

NFKB2, wherein the contiguous sequence of the oligonucleotide is at least 90% complementarity to a human NFKB2 pre-mRNA sequence.

2. The oligonucleotide of embodiment 1, wherein the contiguous nucleotide sequence of the oligonucleotide is complementary to SEQ ID NO 21.

3. The oligonucleotide according to embodiment 1, wherein the contiguous nucleotide sequence of the oligonucleotide is complementary to SEQ ID NO 22.

4. The oligonucleotide of any one of embodiments 1-3, wherein the contiguous nucleotide sequence is complementary to a sub-sequence of the target nucleic acid, wherein the subsequence is selected from the group consisting SEQ ID NO 11, 12, 14, 11, 13, 15, 16, 17, 18, 19, & 20.

5. The oligonucleotide of embodiment 1-4, wherein the oligonucleotide comprises a sequence selected from the group consisting of SEQ ID NO: 1, 2, 3, 4, 5, 6, 7, 8, 9 or 10.

6. The oligonucleotide of embodiment 1-5, comprising one or more modified nucleosides.

7. The oligonucleotide of embodiment 6, wherein the one or more modified nucleosides is a 2′ sugar modified nucleoside.

8. The oligonucleotide of embodiment 7, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

9. The oligonucleotide of any one of embodiments 6 to 8, wherein the one or more modified nucleoside is a LNA nucleoside.

10. The oligonucleotide of any one of embodiments 1-9, where the oligonucleotide comprises at least one modified internucleoside linkage.

11. The oligonucleotide of embodiment 10, wherein the internucleoside linkages within the contiguous nucleotide sequence are phosphorothioate internucleoside linkages.

12. The oligonucleotide of any one of embodiments 1-11, wherein the oligonucleotide is capable of recruiting RNase H.

13. The oligonucleotide of embodiment 12, wherein the oligonucleotide is a gapmer.

14. The oligonucleotide of embodiment 12 or 13, wherein the oligonucleotide is a gapmer of formula 5′-F-G-F′-3′, where region F and F′ independently comprise 1-7 modified nucleosides and G is a region between 6 and 16 nucleosides which are capable of recruiting RNaseH.

15. The oligonucleotide according to any one of embodiments 1-14, wherein said oligonucleotide consists or comprises of an oligonucleotide selected from the group consisting of: CACttaacgtttCGC (SEQ ID NO 1), TGAggtaggtaagtGC (SEQ ID NO 2), TAAcgtttcgcAGC (SEQ ID NO 3), TTGgaatcagacacgT (SEQ ID NO 4), CACaatggaggagTTC (SEQ ID NO 5), GCACttaacgtttCG (SEQ ID NO 6), ACttaacgtttCGCA (SEQ ID NO 7), CCAttcgagaaatCTT (SEQ ID NO 8), CTAtactcagatcCAT (SEQ ID NO 9), and GCCatatcgaaaTCG (SEQ ID NO 10),

wherein capital letters represent LNA nucleosides and lower case letters represent DNA nucleosides, and cytosines are optionally 5-methyl cytosine.

16. The oligonucleotide according to embodiment 15, wherein all LNA nucleotides are beta-D-oxy LNA.

17. The oligonucleotide according to embodiments 15 or 16, wherein all LNA cytosines are 5-methyl cytosine.

18. The oligonucleotide according to any one of embodiments 15-17, wherein all internucleoside linkages present in the indicated sequence are phosphorothioate internucleoside linkages.

19. The oligonucleotide according to any one of embodiments 1-18, wherein the compound is selected from the group consisting of CACttaa^{m}cgtttCGC (SEQ ID NO 1), TGAggtaggtaagtGC (SEQ ID NO 2), TAA^{m}cgttt^{m}cgcAGC (SEQ ID NO 3), TTGgaatcagaca^{m}cgT (SEQ ID NO 4), CACaatggaggagTTC (SEQ ID NO 5), GCACttaa^{m}cgtttCG (SEQ ID NO 6), ACttaa^{m}cgtttCGCA (SEQ ID NO 7), CCAtt^{m}cgagaaatCTT (SEQ ID NO 8), CTAtactcagatcCAT (SEQ ID NO 9), and GCCatat^{m}cgaaaTCG (SEQ ID NO 10), wherein capital letters represent beta-D-oxy LNA nucleosides, all LNA cytosines are 5-methyl cytosine, lower case letters are DNA nucleosides, ^{m}c indicates a 5-methyl cytosinse DNA nucleoside, and all internucleoside linkages are phosphorothioate internucleoside linkages.

20. A conjugate comprising the oligonucleotide according to any one of embodiments 1-19, and at least one conjugate moiety covalently attached to said oligonucleotide.

21. A pharmaceutical composition comprising the oligonucleotide of embodiment 1-19 or the conjugate of embodiment 20 and a pharmaceutically acceptable diluent, solvent, carrier, salt and/or adjuvant.

22. An in vivo or in vitro method for modulating NF-κB2 expression in a target cell which is expressing NF-κB2 said method comprising administering an oligonucleotide of any one of embodiments 1-19, the conjugate according to embodiment 20, or the pharmaceutical composition of embodiment 21 in an effective amount to said cell.

23. A method for treating or preventing a disease comprising administering a therapeutically or prophylactically effective amount of an oligonucleotide of any one of embodiments 1-19 or the conjugate according to embodiment 20 or the pharmaceutical composition of embodiment 21 to a subject suffering from or susceptible to the disease.

24. The method of embodiment 23, wherein the disease is selected from the group consisting of cancer, inflammation and inflammatory disorders, and autoimmune diseases.

25. The method according to embodiment 24, wherein the disease is selected from the group consisting of multiple sclerosis, Crohn's disease and rheumatoid arthritis.

26. The oligonucleotide of any one of embodiments 1-19 or the conjugate according to embodiment 20 or the pharmaceutical composition of embodiment 21 for use in medicine.

27. The oligonucleotide of any one of embodiments 1-19 or the conjugate according to embodiment 20 or the pharmaceutical composition of embodiment 21 for use in the treatment or prevention of cancer, inflammation and inflammatory disorders, and autoimmune diseases.

28. The use of the oligonucleotide of embodiment 1-19 or the conjugate according to embodiment 20 or the pharmaceutical composition of embodiment 21, for the preparation of a medicament for treatment or prevention of cancer, inflammation and inflammatory disorders, and autoimmune diseases.

29. The oligonucleotide or use according to any one of embodiments 26-28, wherein the oligonucleotide is for use in the treatment of a disease selected from the group consisting of multiple sclerosis, rheumatoid arthritis, ulcerative colitis or B cell lymphomas

### EXAMPLES

The work reported herein has received funding from the European Union Seventh Framework Programme [FP7-2007-2013] under grant agreement “HEALTH-F2-2013-602114” (Athero-B-Cell).

### Materials and Methods

Oligonucleotide Synthesis

Oligonucleotide synthesis is generally known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

Elongation of the Oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphordiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

Purification by RP-HPLC:

The crude compounds are purified by preparative RP-HPLC on a Phenomenex Jupiter C18 10μ 150×10 mm column. 0.1 M ammonium acetate pH 8 and acetonitrile is used as buffers at a flow rate of 5 mL/min. The collected fractions are lyophilized to give the purified compound typically as a white solid.

Abbreviations:

DCI: 4,5-Dicyanoimidazole

DCM: Dichloromethane

DMF: Dimethylformamide

DMT: 4,4′-Dimethoxytrityl

THF: Tetrahydrofurane

Bz: Benzoyl

Ibu: Isobutyryl

RP-HPLC: Reverse phase high performance liquid chromatography

Tₘ Assay:

Oligonucleotide and RNA target (phosphate linked, PO) duplexes are diluted to 3 mM in 500 ml RNase-free water and mixed with 500 ml 2× Tₘ-buffer (200 mM NaCl, 0.2 mM EDTA, 20 mM Naphosphate, pH 7.0). The solution is heated to 95° C. for 3 min and then allowed to anneal in room temperature for 30 min. The duplex melting temperatures (Tₘ) is measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

### Example 1

Testing In Vitro Potency and Efficacy of Selected Oligonucleotides Targeting Mouse Nfkb-subunit mRNA in RAW264.7 Cells in a Dose Response Curve.

RAW 264.7 cell line was purchased from ATCC and maintained as recommended by the supplier in a humidified incubator at 37° C. with 5% CO2. For assays, 2500 cells/well were seeded in a 96 multi well plate in culture media. Cells were incubated for 24 hours before addition of oligonucleotides dissolved in PBS. Concentration of oligonucleotides: from 50 μM, 1:1 dilution in 8 steps. Three days after addition of oligonucleotides, the cells were harvested. RNA was extracted using the PureLink Pro 96 RNA Purification kit (Thermo Fisher Scientific) according to the manufacturer's instructions and eluated in 50 μl water. The RNA was subsequently diluted 10 times with DNase/RNase free Water (Gibco) and heated to 90° C. for one minute.

For gene expressions analysis, One Step RT-qPCR was performed using gScript™ XLT One-Step RT-qPCR Tough Mix®, Low ROX™ (Quantabio) in a duplex set up. The following TaqMan primer assays were used for qPCR: Nfkb1, Mm00476361_ml; Nfkb2, Mm00479810_g1; Rela Mm00501346_m1; Relb, Mm00485664_ml; or Rel, Mm01239661_m1 (FAM-MGB); each combined with endogenous control Gapdh, Mm99999915_g1 (VIC-MGB). All primer sets were purchased from Thermo Fisher Scientific. IC₅₀ determinations were performed in Graph Pad Prism6. The relative mRNA levels at treatment with 50 μM oligonucleotide is shown in the table as percent of control (PBS).

| | | | | | | mRNA |
|---|---|---|---|---|---|---|
| SEQ | | | CMP | | | level |
| ID | | | ID | | IC50 | at Max |
| NO | Target | Motif | NO | Compound | [μM] | KD |
| 23 | Nfkb2 | agatttcgat | M1, 1 | AGATttcga | 2, 5 | 38 |
| | | tagac | | ttagAC | | |
| 24 | Relb | tagaattgaa | M2, 1 | TAGAattga | 1, 1 | 13 |
| | | gttaaa | | agtTAAA | | |
| 25 | Rela | ataactgtgt | M3, 1 | ATaactgtg | 2, 7 | 41 |
| | | tttc | | ttTTC | | |
| | Rel | | | | 3, 5 | 65 |

### Example 2

### Mouse In Vivo Efficacy and Tolerance Study, 16 Days of Treatment, Intravenous Injection (Tail Vein).

Animals

Experiment was performed on female C57BL/6JBom mice (Taconic). Five animals were included in each group of the study, including a saline control group.

Compounds and Dosing Procedures

Animals were injected intravenously (tail vein) with 15 mg/kg compound at day 0, 3, 7, 10, 14 until the study was terminated at day 16.

Euthanasia

At the end of the study (day 16) all mice were euthanized with CO₂ before tissue samples of liver, kidney and mesenteric lymph node were dissected and snap frozen.

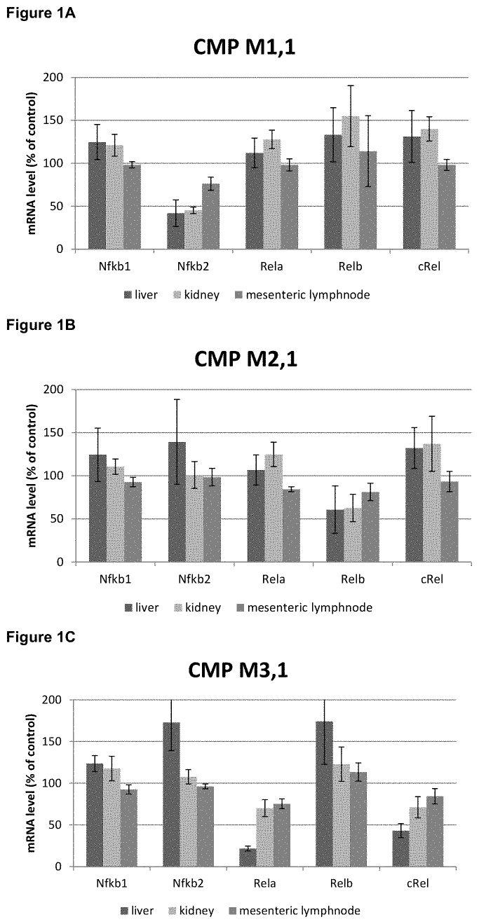

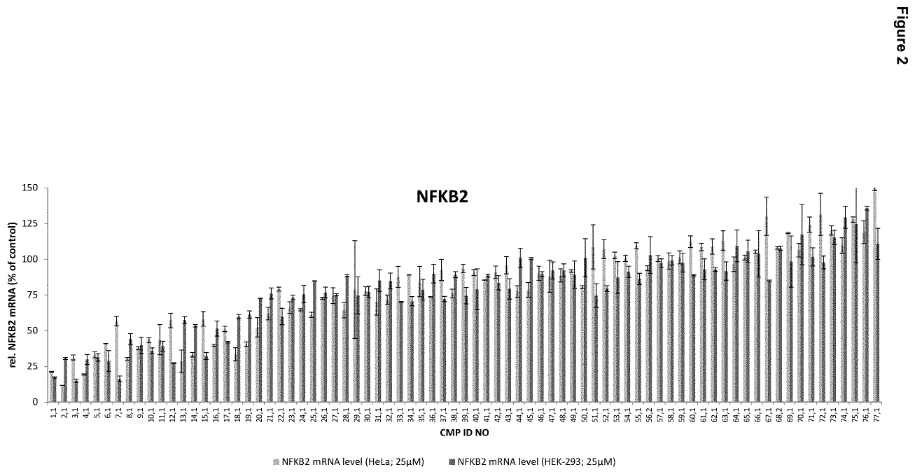

Quantification of Nfkb-subunit RNA expression (FIG. 1A, 1B and 1C)

Tissue samples were kept frozen until lysed in MagNA Pure LC RNA Isolation Tissue Lysis Buffer (Product No. 03604721001, Roche) and RNA extraction continued using the MagNA Pure 96 Cellular RNA Large Volume Kit (Product No. 05467535001, Roche) on a MagNA Pure 96 Instrument (Roche) according to the user's manual and RNA diluted to 5 ng/μl in water.

For gene expressions analysis, One Step RT-qPCR was performed using gScript™ XLT One-Step RT-qPCR Tough Mix®, Low ROX™ (Quantabio) in a duplex set up. The following TaqMan primer assays were used for qPCR: Nfkb1, Mm00476361_ml; Nfkb2, Mm00479810_g1; Rela Mm00501346_m1; Relb, Mm00485664_ml; or Rel, Mm01239661_m1 (FAM-MGB); each combined with endogenous control Gapdh, Mm99999915_g1 (VIC-MGB). All primer sets were purchased from Thermo Fisher Scientific. The relative mRNA expression levels are shown as % of control (PBS-treated animals).

### Example 3

Testing In Vitro Efficacy of Antisense Oligonucleotides Targeting Human NFKB2 mRNA in HEK293 and HeLa Cell Lines at Single Dose Concentration.

NFKB2 nuclear factor kappa B subunit 2 [Homo sapiens (human)]

Also known as: p52; p100; H2TF1; LYT10; CVID10; LYT-10; NF-kB2; p49/p100

| Assembly | Chr | Location |
|---|---|---|
| GRCh38.p7 | 10 | NC_000010.11 |
| (GCF_000001405.33) | | (102394110..102402529) |

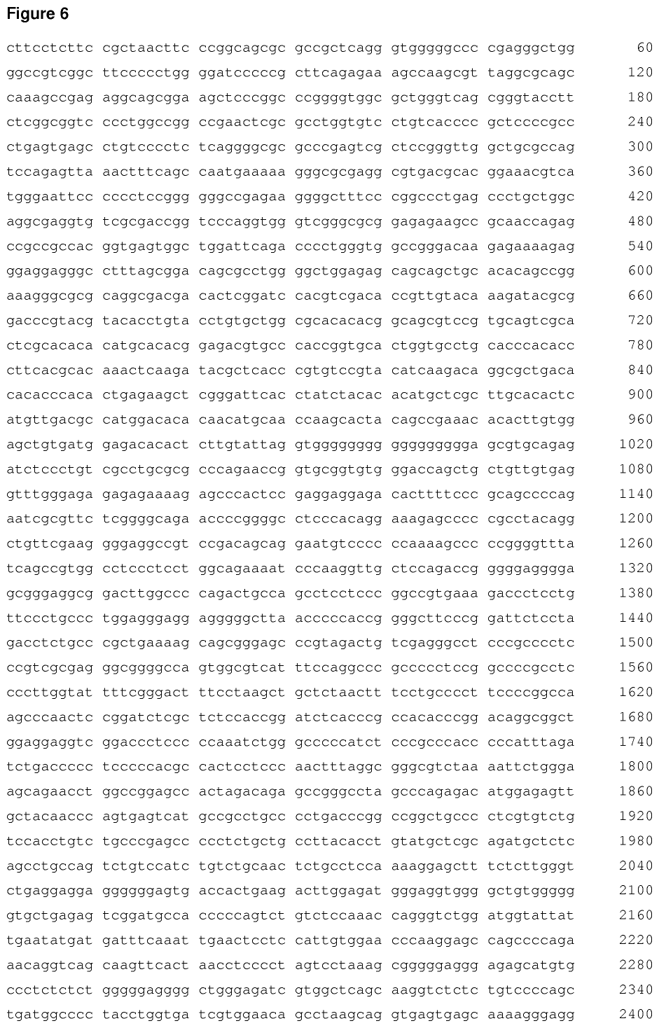

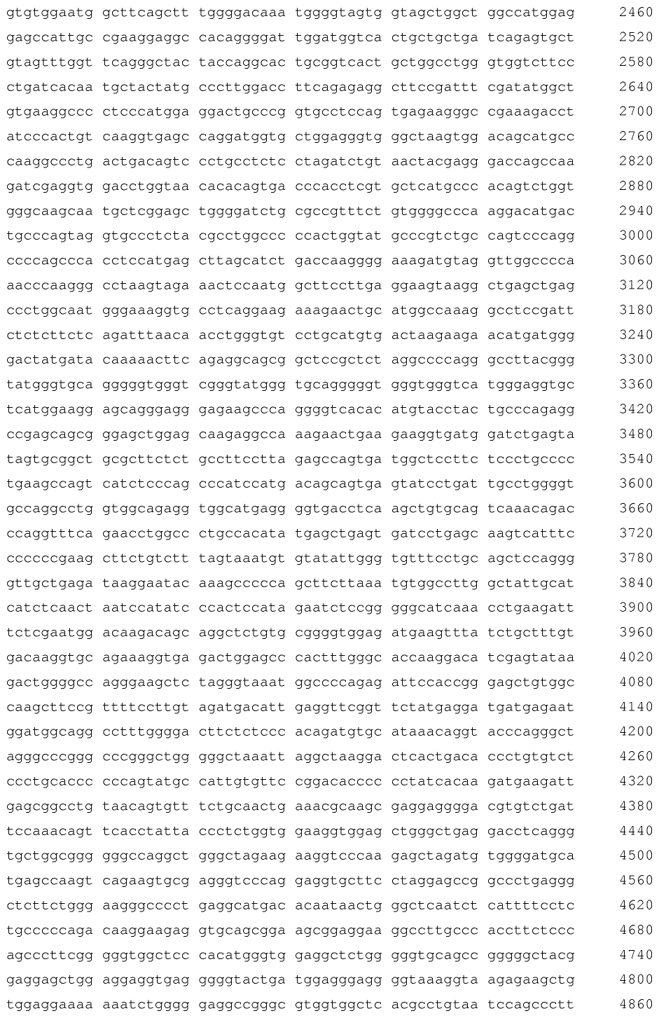

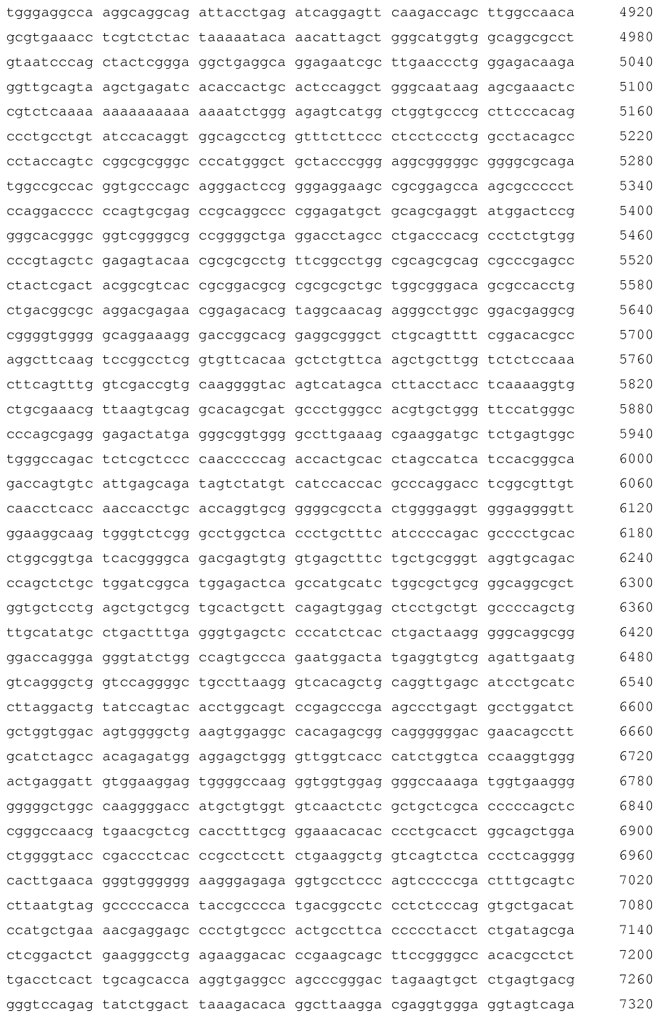

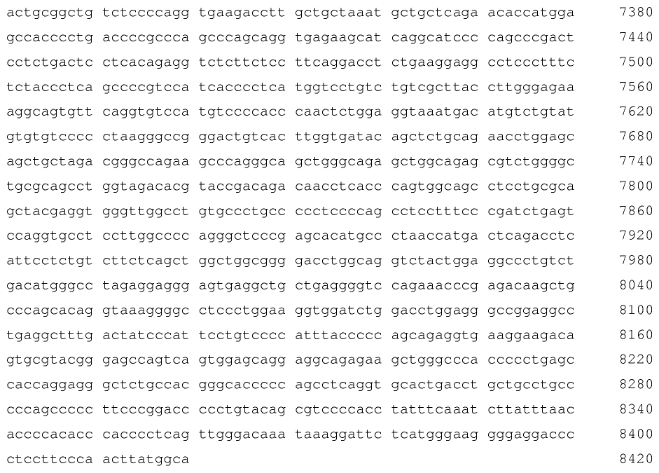

The Human NFKB2 pre-mRNA sequence is provided as SEQ ID NO 21 (FIG. 6).

HEK-293 and HeLa cell lines were purchased from ATCC and maintained as recommended by the supplier in a humidified incubator at 37° C. with 5% CO₂. For assays, 3500 cells/well (HEK-293) or 3000 cells/well (HeLa) were seeded in a 96 multi well plate in culture media. Cells were incubated for 24 hours before addition of oligonucleotides dissolved in PBS. Final concentration of oligonucleotides: 25 μM. Three days after addition of oligonucleotides, the cells were harvested. RNA was extracted using the PureLink Pro 96 RNA Purification kit (Thermo Fisher Scientific) according to the manufacturer's instructions and eluated in 50 μwater. The RNA was subsequently diluted 10 times with DNase/RNase free Water (Gibco) and heated to 90° C. for one minute.

For gene expressions analysis, One Step RT-qPCR was performed using gScript™ XLT One-Step RT-qPCR ToughMix®, Low ROX™ (Quantabio) in a duplex set up. The following TaqMan primer assays were used for qPCR: NFKB2, Hs01028901_g1 (FAM-MGB) and endogenous control GAPDH, Hs99999905_m1 (VIC-MGB). All primer sets were purchased from Thermo Fisher Scientific. The relative NFKB2 mRNA expression level in Table 1 is shown as percent of control (PBS-treated cells). A total of 77 compounds were designed at a length of 15-16 nucleotides with varying LNA patterns (3×3; 2×4; 4×2; 3×2; 2×3) across SEQ ID NO 21. A waterfall plot of relative NFKB2 expression in two cell lines in shown in FIG. 2.

TABLE 1

| | | | | Rel. | Rel. |
|---|---|---|---|---|---|
| | | | | mRNA | mRNA |
| | | | | level | level |
| SEQ | | CMP | | HEK-293 | HeLa |
| ID | | ID | | at | at |
| NO | Motif | NO | Compound | 25 μM | 25 μM |
| 1 | cacttaacgtttcgc | 1, 1 | CACttaa^{m}c | 17 | 21 |
| | | | gtttCGC | | |
| 2 | tgaggtaggtaagtgc | 2, 1 | TGAggtagg | 31 | 12 |
| | | | taagtGC | | |
| 3 | taacgtttcgcagc | 3, 1 | TAA^{m}cgttt | 15 | 31 |
| | | | ^{m}cgcAGC | | |
| 4 | ttggaatcagacacgt | 4, 1 | TTGgaatca | 30 | 19 |
| | | | gaca^{m}cgT | | |
| 5 | cacaatggaggagttc | 5, 1 | CACaatgga | 31 | 33 |
| | | | ggagTTC | | |
| 6 | gcacttaacgtttcg | 6, 1 | GCACttaa^{m} | 29 | 41 |
| | | | cgtttCG | | |
| 7 | acttaacgtttcgca | 7, 1 | ACttaa^{m}cg | 16 | 57 |
| | | | tttCGCA | | |
| 8 | ccattcgagaaatctt | 8, 1 | CCAtt^{m}cga | 44 | 30 |
| | | | gaaatCTT | | |
| 9 | ctatactcagatccat | 9, 1 | CTAtactca | 40 | 38 |
| | | | gatcCAT | | |
| 10 | gccatatcgaaatcg | 10, 1 | GCCatat^{m}c | 36 | 43 |
| | | | gaaaTCG | | |

For Compounds: Capital letters represent LNA nucleosides (beta-D-oxy LNA nucleosides were used), all LNA cytosines are 5-methyl cytosine, lower case letters represent DNA nucleosides, DNA cytosines preceded with a superscript ^{m} represents a 5-methyl C-DNA nucleoside. All internucleoside linkages are phosphorothioate internucleoside linkages.

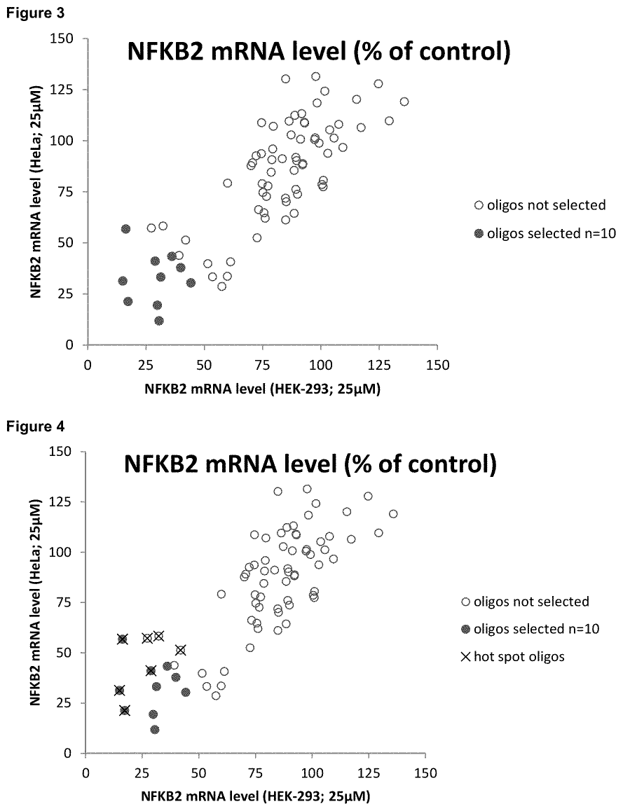

The data obtained from the two cell lines is shown in FIG. 3, which illustrates that the above compounds were particularly effective in both cell lines in targeting human NFKB2, as compared to a library of other compounds targeting human NFKB2. Each of the 10 sequences aligned to the following regions of the NFKB2 transcript, illustrated in FIG. 4, referred to as hotspot regions A, B, C, D, E, F, G, H, I, & J:

| | Hotspot | | | | |
|---|---|---|---|---|---|
| | Region- | | | | |
| | NFKB2 | | | | |
| | pre- | | | | Target |
| Hotspot | mRNA | | | | SEQ |
| Region | position | Se- | Com- | Target | ID |
| (NFKB2) | (start) | quences | pounds | Sequence | NO |
| A | 5823 | 1 | 1, 1 | GCGAAACGT | 11 |
| | | | | TAAGTG | |
| B | 5798 | 2 | 2, 1 | GCACTTACC | 12 |
| | | | | TACCTCA | |
| C | 5820 | 3 | 3, 1 | GCTGCGAAA | 13 |
| | | | | CGTTA | |
| D | 4370 | 4 | 4, 1 | ACGTGTCTG | 14 |
| | | | | ATTCCAA | |
| E | 2182 | 5 | 5, 1 | GAACTCCTC | 15 |
| | | | | CATTGTG | |
| F | 5824 | 6 | 6, 1 | CGAAACGTT | 16 |
| | | | | AAGTGC | |
| G | 5822 | 7 | 7, 1 | TGCGAAACG | 17 |
| | | | | TTAAGT | |
| H | 3895 | 8 | 8, 1 | AAGATTTCT | 18 |
| | | | | CGAATGG | |
| I | 3468 | 9 | 9, 1 | ATGGATCTG | 19 |
| | | | | AGTATAG | |
| J | 2625 | 10 | 10, 1 | CGATTTCGA | 20 |
| | | | | TATGGC | |

SEQ ID NO 22=GCACTTACCTACCTCAAAAGGTGCTGCGAAACGTTAAGTGC, and encompasses both SEQ ID NO 11, 13, 16 & 17. Compounds 1, 2, 3, 6 & 7 target SEQ ID NO 22. SEQ ID NO 22 is therefore a preferred hotspot region for antisense targeting.

### Example 4

Testing in vitro potency and efficacy of selected oligonucleotides targeting human NFKB2 mRNA in HEK-293 and HeLa cell lines in a dose response curve.

HEK-293 cell line and HeLa cell line was described in Example 1. The assay was performed as described in Example 1. Concentration of oligonucleotides: from 50 μM, 1:1 dilution in 8 steps. Three days after addition of oligonucleotides, the cells were harvested. RNA extraction and duplex One Step RT-qPCR were performed as described in Example3. n=2 biological replicates per each cell line. IC₅₀ determinations were performed in Graph Pad Prism6. The relative NFKB2 mRNA level at treatment with 50 μM oligonucleotide is shown in the table as percent of control (PBS).

| | | | | | mRNA |
|---|---|---|---|---|---|
| | | | mRNA | | level at |
| | | | level at | | Max |
| | | | Max | IC50 | KD in |
| SEQ | CMP | IC50 | KD in | HEK- | HEK- |
| ID NO | ID NO | HeLa | HeLa | 293 | 293 |
| 1 | 1.1 | 4.4 | 36 | 5.8 | 13 |
| 2 | 2.1 | 1.9 | 19 | 2.1 | 29 |
| 3 | 3.1 | 2.9 | 41 | 2.8 | 12 |
| 4 | 4.1 | 2.1 | 22 | 2.4 | 15 |
| 5 | 5.1 | 3.3 | 48 | 2.0 | 25 |
| 6 | 6.1 | 2.2 | 50 | 2.6 | 21 |
| 7 | 7.1 | 1.7 | 60 | 1.8 | 11 |
| 8 | 8.1 | 2.4 | 33 | 4.0 | 30 |
| 9 | 9.1 | 4.4 | 35 | 8.7 | 20 |
| 10 | 10.1 | 6.4 | 57 | 3.5 | 30 |

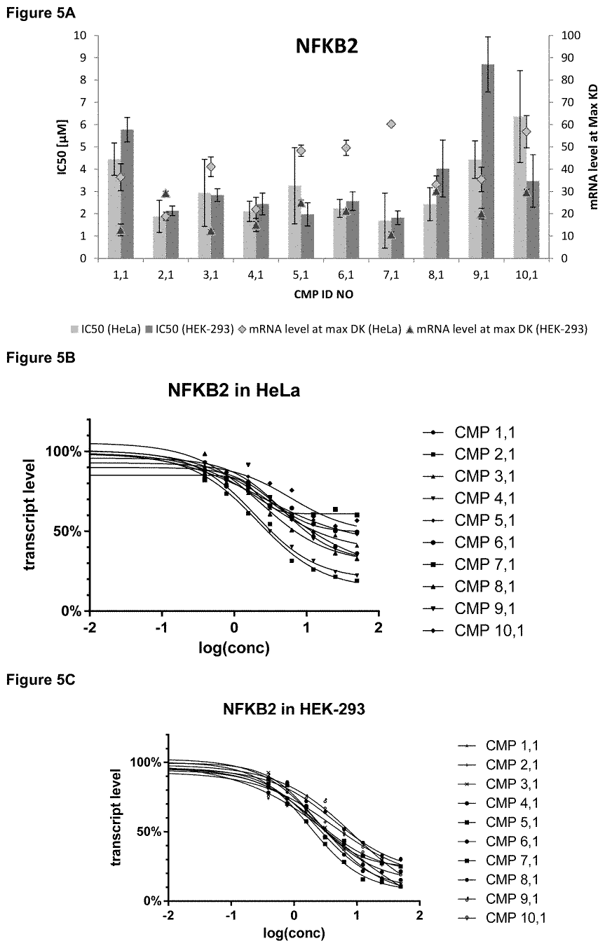

The data and IC₅₀ curves are shown in FIGS. 5A, 5B and 5C.

1. An LNA gapmer antisense oligonucleotide of 12 to 30 contiguous nucleotides in length, targeting RELB, wherein a contiguous nucleotide sequence of the oligonucleotide is at least 90% complementary to the human RELB pre-mRNA sequence, such as SEQ ID NO: 21, wherein the LNA gapmer antisense oligonucleotide is capable of inhibiting the expression of RELB in a cell which is expressing RELB; or a pharmaceutically acceptable salt thereof.

2. The LNA gapmer antisense oligonucleotide of claim 1, wherein the contiguous nucleotide sequence of the oligonucleotide is complementary to a RELB intron sequence.

3. The LNA gapmer antisense oligonucleotide according to claim 1, wherein the contiguous nucleotide sequence of the oligonucleotide is complementary to intron region i5 or i4 of SEQ ID NO: 21.

4. The LNA gapmer antisense oligonucleotide of claim 1, wherein the contiguous nucleotide sequence is complementary to a sub-sequence of a target nucleic acid, wherein the subsequence is selected from the group consisting of SEQ ID NO: 11, 12, 13, 14, 15, 16, 17, 18, 19, & 20.

5. The LNA gapmer antisense oligonucleotide of claim 1, wherein the oligonucleotide comprises a sequence selected from the group consisting of SEQ ID NO: 1, 2, 3, 4, 5, 6, 7, 8, 9 or 10.

6. The LNA gapmer antisense oligonucleotide of claim 1, wherein the LNA gapmer antisense oligonucleotide comprises a gapmer region of a formula 5′-F-G-F′-3′, where region F and F′ independently comprise 1-7 modified nucleosides and G is a region of between 6 and 16 nucleosides which are capable of recruiting RNaseH.

7. The LNA gapmer antisense oligonucleotide according to claim 1, wherein said oligonucleotide consists or comprises an oligonucleotide selected from the group consisting of: TCggaatacagcAGG (SEQ ID NO: 1), GTGaatagaggtagGT (SEQ ID NO: 2), GTGgagaatcaggTG (SEQ ID NO: 3), ACAgagttagacacCA (SEQ ID NO: 4), TCAtaatactcggtGC (SEQ ID NO: 5), CAGagttagacacCA (SEQ ID NO: 6), ACGgcattaacaagGA (SEQ ID NO: 7), TGAgataggacaacCA (SEQ ID NO: 8), CAgagttagacacCAT (SEQ ID NO: 9), and CATAatactcggtgCT (SEQ ID NO: 10), wherein capital letters represent LNA nucleosides and lower case letters represent DNA nucleosides, and cytosines are optionally 5-methyl cytosine.

8. The LNA gapmer antisense oligonucleotide according to claim 7, wherein all LNA nucleotides are beta-D-oxy LNA.

9. The LNA gapmer antisense oligonucleotide according to claim 7, wherein all LNA cytosines are 5-methyl cytosine.

10. The LNA gapmer antisense oligonucleotide according to claim 5, wherein all internucleoside linkages present in the gapmer region of the LNA gapmer antisense oligonucleotide compound are phosphorothioate internucleoside linkages.

11. The LNA gapmer antisense oligonucleotide according to claim 1, wherein the compound is selected from the group consisting of TCggaatacagcAGG (SEQ ID NO: 1), GTGaatagaggtagGT (SEQ ID NO: 2), GTGgagaatcaggTG (SEQ ID NO: 3), ACAgagttagacacCA (SEQ ID NO: 4), TCAtaatactmcggtGC (SEQ ID NO: 5), CAGagttagacacCA (SEQ ID NO: 6), ACGgcattaacaagGA (SEQ ID NO: 7), TGAgataggacaacCA (SEQ ID NO: 8), CAgagttagacacCAT (SEQ ID NO: 9), and CATAatactmcggtgCT (SEQ ID NO: 10), wherein capital letters represent beta-D-oxy LNA nucleosides, all LNA cytosines are 5-methyl cytosine, lower case letters are DNA nucleosides, mc indicates a 5-methyl cytosine DNA nucleoside, and all internucleoside linkages are phosphorothioate internucleoside linkages.

12. A conjugate comprising the LNA gapmer antisense oligonucleotide according to claim 1, and at least one conjugate moiety covalently attached to said oligonucleotide.

13. A pharmaceutical composition comprising the LNA gapmer antisense oligonucleotide of claim 1 and a pharmaceutically acceptable diluent, solvent, carrier, salt and/or adjuvant.

14. An in vitro method for modulating RELB expression in a target cell which is expressing RelB said method comprising administering an LNA gapmer antisense oligonucleotide of claim 1 in an effective amount to said cell.

15. The LNA gapmer antisense oligonucleotide of claim 1 for use in medicine.

16. The LNA gapmer antisense oligonucleotide of claim 1 for use in the treatment or prevention of cancer, inflammation and inflammatory disorders, and autoimmune diseases.

17. The use of the LNA gapmer antisense oligonucleotide of claim 1, for the preparation of a medicament for treatment or prevention of cancer, inflammation and inflammatory disorders, and autoimmune diseases.

18. The LNA gapmer antisense oligonucleotide of claim 1, wherein the oligonucleotide is for use in the treatment of a disease selected from the group consisting of prostate cancer, breast cancer, multiple sclerosis, colitis, Crohn's disease and rheumatoid arthritis.


SEQUENCE LISTING (25 sequences)

SEQ ID NO: 1 - DNA, 15 bp, Artificial Sequence
  oligonucleotide or target sequence
cacttaacgtttcgc

SEQ ID NO: 2 - DNA, 16 bp, Artificial Sequence
  oligonucleotide or target sequence
tgaggtaggtaagtgc

SEQ ID NO: 3 - DNA, 14 bp, Artificial Sequence
  oligonucleotide or target sequence
taacgtttcgcagc

SEQ ID NO: 4 - DNA, 16 bp, Artificial Sequence
  oligonucleotide or target sequence
ttggaatcagacacgt

SEQ ID NO: 5 - DNA, 16 bp, Artificial Sequence
  oligonucleotide or target sequence
cacaatggaggagttc

SEQ ID NO: 6 - DNA, 15 bp, Artificial Sequence
  oligonucleotide or target sequence
gcacttaacgtttcg

SEQ ID NO: 7 - DNA, 15 bp, Artificial Sequence
  oligonucleotide or target sequence
acttaacgtttcgca

SEQ ID NO: 8 - DNA, 16 bp, Artificial Sequence
  oligonucleotide or target sequence
ccattcgagaaatctt

SEQ ID NO: 9 - DNA, 16 bp, Artificial Sequence
  oligonucleotide or target sequence
ctatactcagatccat

SEQ ID NO: 10 - DNA, 15 bp, Artificial Sequence
  oligonucleotide or target sequence
gccatatcgaaatcg

SEQ ID NO: 11 - DNA, 15 bp, Artificial Sequence
  oligonucleotide or target sequence
gcgaaacgttaagtg

SEQ ID NO: 12 - DNA, 16 bp, Artificial Sequence
  oligonucleotide or target sequence
gcacttacctacctca

SEQ ID NO: 13 - DNA, 14 bp, Artificial Sequence
  oligonucleotide or target sequence
gctgcgaaacgtta

SEQ ID NO: 14 - DNA, 16 bp, Artificial Sequence
  oligonucleotide or target sequence
acgtgtctgattccaa

SEQ ID NO: 15 - DNA, 16 bp, Artificial Sequence
  oligonucleotide or target sequence
gaactcctccattgtg

SEQ ID NO: 16 - DNA, 15 bp, Artificial Sequence
  oligonucleotide or target sequence
cgaaacgttaagtgc

SEQ ID NO: 17 - DNA, 15 bp, Artificial Sequence
  oligonucleotide or target sequence
tgcgaaacgttaagt

SEQ ID NO: 18 - DNA, 16 bp, Artificial Sequence
  oligonucleotide or target sequence
aagatttctcgaatgg

SEQ ID NO: 19 - DNA, 16 bp, Artificial Sequence
  oligonucleotide or target sequence
atggatctgagtatag

SEQ ID NO: 20 - DNA, 15 bp, Artificial Sequence
  oligonucleotide or target sequence
cgatttcgatatggc

SEQ ID NO: 21 - DNA, 8420 bp, homo sapiens
cttcctcttccgctaacttcccggcagcgcgccgctcagggtgggggccccgagggctggggccgtcggcttccccctggggatcccccgcttcagagaaagccaagcgttaggcgcagccaaagccgagaggcagcggaagctcccggcccggggtggcgctgggtcagcgggtaccttctcggcggtcccctggccggccgaactcgcgcctggtgtcctgtcaccccgctccccgccctgagtgagcctgtcccctctcaggggcgcgcccgagtcgctccgggttggctgcgccagtccagagttaaactttcagccaatgaaaaagggcgcgaggcgtgacgcacggaaacgtcatgggaattcccccctccggggggccgagaaggggctttcccggccctgagccctgctggcaggcgaggtgtcgcgaccggtcccaggtgggtcgggcgcggagagaagccgcaaccagagccgccgccacggtgagtggctggattcagacccctgggtggccgggacaagagaaaagagggaggagggcctttagcggacagcgcctggggctggagagcagcagctgcacacagccggaaagggcgcgcaggcgacgacactcggatccacgtcgacaccgttgtacaaagatacgcggacccgtacgtacacctgtacctgtgctggcgcacacacggcagcgtccgtgcagtcgcactcgcacacacatgcacacggagacgtgcccaccggtgcactggtgcctgcacccacacccttcacgcacaaactcaagatacgctcacccgtgtccgtacatcaagacaggcgctgacacacacccacactgagaagctcgggattcacctatctacacacatgctcgcttgcacactcatgttgacgccatggacacacaacatgcaaccaagcactacagccgaaacacacttgtggagctgtgatggagacacactcttgtattaggtgggggggggggggggggagcgtgcagagatctccctgtcgcctgcgcgcccagaaccggtgcggtgtgggaccagctgctgttgtgaggtttgggagagagagaaaagagcccactccgaggaggagacacttttcccgcagccccagaatcgcgttctcggggcagaaccccggggcctcccacaggaaagagcccccgcctacaggctgttcgaaggggaggccgtccgacagcaggaatgtccccccaaaagcccccggggtttatcagccgtggcctccctcctggcagaaaatcccaaggttgctccagaccgggggaggggagcgggaggcggacttggccccagactgccagcctcctcccggccgtgaaagaccctcctgttccctgccctggagggaggagggggcttaacccccaccggggcttcccggattctcctagacctctgcccgctgaaaagcagcgggagcccgtagactgtcgagggcctcccgcccctcccgtcgcgagggcggggccagtggcgtcatttccaggcccgccccctccggccccgcctccccttggtattttcgggactttcctaagctgctctaactttcctgccccttccccggccaagcccaactccggatctcgctctccaccggatctcacccgccacacccggacaggcggctggaggaggtcggaccctcccccaaatctgggcccccatctcccgcccacccccatttagatctgaccccctcccccacgccactcctcccaactttaggcgggcgtctaaaattctgggaagcagaacctggccggagccactagacagagccgggcctagcccagagacatggagagttgctacaacccagtgagtcatgccgcctgcccctgacccggccggctgcccctcgtgtctgtccacctgtctgcccgagccccctctgctgccttacacctgtatgctcgcagatgctctcagcctgccagtctgtccatctgtctgcaactctgcctccaaaaggagctttctcttgggtctgaggaggaggggggagtgaccactgaagacttggagatgggaggtggggctgtggggggtgctgagagtcggatgccacccccagtctgtctccaaaccagggtctggatggtattattgaatatgatgatttcaaattgaactcctccattgtggaacccaaggagccagccccagaaacaggtcagcaagttcactaacctcccctagtcctaaagcgggggagggagagcatgtgccctctctctgggggaggggctgggagatcgtggctcagcaaggtctctctgtccccagctgatggcccctacctggtgatcgtggaacagcctaagcaggtgagtgagcaaaagggagggtgtggaatggcttcagctttggggacaaatggggtagtggtagctggctggccatggaggagccattgccgaaggaggccacaggggattggatggtcactgctgctgatcagagtgctgtagtttggttcagggctactaccaggcactgcggtcactgctggcctgggtggtcttccctgatcacaatgctactatgcccttggaccttcagagaggcttccgatttcgatatggctgtgaaggcccctcccatggaggactgcccggtgcctccagtgagaagggccgaaagacctatcccactgtcaaggtgagccaggatggtgctggagggtgggctaagtggacagcatgcccaaggccctgactgacagtccctgcctctcctagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccagtaggtgccctctacgcctggcccccactggtatgcccgtctgccagtcccaggccccagcccacctccatgagcttagcatctgaccaaggggaaagatgtaggttggccccaaacccaagggcctaagtagaaactccaatggcttccttgaggaagtaaggctgagctgagccctggcaatgggaaaggtgcctcaggaagaaagaactgcatggccaaaggcctccgattctctcttctcagatttaacaacctgggtgtcctgcatgtgactaagaagaacatgatggggactatgatacaaaaacttcagaggcagcggctccgctctaggccccagggccttacgggtatgggtgcagggggtgggtcgggtatgggtgcagggggtgggtgggtcatgggaggtgctcatggaaggagcagggagggagaagcccaggggtcacacatgtacctactgcccagaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcagtgagtatcctgattgcctggggtgccaggcctggtggcagaggtggcatgaggggtgacctcaagctgtgcagtcaaacagacccaggtttcagaacctggccctgccacatatgagctgagtgatcctgagcaagtcatttcccccccgaagcttctgtctttagtaaatgtgtatattgggtgtttcctgcagctccaggggttgctgagataaggaatacaaagcccccagcttcttaaatgtggccttggctattgcatcatctcaactaatccatatcccactccatagaatctccgggggcatcaaacctgaagatttctcgaatggacaagacagcaggctctgtgcggggtggagatgaagtttatctgctttgtgacaaggtgcagaaaggtgagactggagcccactttgggcaccaaggacatcgagtataagactggggccagggaagctctagggtaaatggccccagagattccaccgggagctgtggccaagcttccgttttccttgtagatgacattgaggttcggttctatgaggatgatgagaatggatggcaggcctttggggacttctctcccacagatgtgcataaacaggtacccagggctagggcccgggcccgggctgggggctaaattaggctaaggactcactgacaccctgtgtctccctgcaccccccagtatgccattgtgttccggacacccccctatcacaagatgaagattgagcggcctgtaacagtgtttctgcaactgaaacgcaagcgaggaggggacgtgtctgattccaaacagttcacctattaccctctggtggaaggtggagctgggctgaggacctcagggtgctggcggggggccaggctgggctagaagaaggtcccaagagctagatgtggggatgcatgagccaagtcagaagtgcgagggtcccaggaggtgcttcctaggagccggccctgagggctcttctgggaagggcccctgaggcatgacacaataactgggctcaatctcattttcctctgcccccagacaaggaagaggtgcagcggaagcggaggaaggccttgcccaccttctcccagcccttcgggggtggctcccacatgggtggaggctctgggggtgcagccgggggctacggaggagctggaggaggtgagggggtactgatggagggaggggtaaaggtaagagaagctgtggaggaaaaaaatctgggggaggccgggcgtggtggctcacgcctgtaatccagccctttgggaggccaaggcaggcagattacctgagatcaggagttcaagaccagcttggccaacagcgtgaaacctcgtctctactaaaaatacaaacattagctgggcatggtggcaggcgcctgtaatcccagctactcgggaggctgaggcaggagaatcgcttgaaccctgggagacaagaggttgcagtaagctgagatcacaccactgcactccaggctgggcaataagagcgaaactccgtctcaaaaaaaaaaaaaaaaaatctgggagagtcatggctggtgcccgcttcccacagccctgcctgtatccacaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccaagcgccccctccaggaccccccagtgcgagccgcaggccccggagatgctgcagcgaggtatggactccggggcacgggcggtcggggcgccggggctgaggacctagccctgacccacgccctctgtggcccgtagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacgtaggcaacagagggcctggcggacgaggcgcggggtgggggcaggaaagggaccggcacggaggcgggctctgcagttttcggacacgccaggcttcaagtccggcctcggtgttcacaagctctgttcaagctgcttggtctctccaaacttcagtttggtcgaccgtgcaaggggtacagtcatagcacttacctacctcaaaaggtgctgcgaaacgttaagtgcaggcacagcgatgccctgggccacgtgctgggttccatgggccccagcgagggagactatgagggcggtggggccttgaaagcgaaggatgctctgagtggctgggccagactctcgctccccaacccccagaccactgcacctagccatcatccacgggcagaccagtgtcattgagcagatagtctatgtcatccaccacgcccaggacctcggcgttgtcaacctcaccaaccacctgcaccaggtgcgggggcgcctactggggaggtgggaggggttggaaggcaagtgggtctcgggcctggctcaccctgctttcatccccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggtgagctccccatctcacctgactaagggggcaggcggggaccagggagggtatctggccagtgcccagaatggactatgaggtgtcgagattgaatggtcagggctggtccaggggctgccttaagggtcacagctgcaggttgagcatcctgcatccttaggactgtatccagtacacctggcagtccgagcccgaagccctgagtgcctggatctgctggtggacagtggggctgaagtggaggccacagagcggcaggggggacgaacagccttgcatctagccacagagatggaggagctggggttggtcacccatctggtcaccaaggtgggactgaggattgtggaaggagtggggccaagggtggtggaggggccaaagatggtgaaggggggggctggccaaggggaccatgctgtggtgtcaactctcgctgctcgcacccccagctccgggccaacgtgaacgctcgcacctttgcgggaaacacacccctgcacctggcagctggactggggtacccgaccctcacccgcctccttctgaaggctggtcagtctcaccctcaggggcacttgaacagggtggggggaagggagagaggtgcctcccagtcccccgactttgcagtccttaatgtaggcccccaccataccgccccatgacggcctccctctcccaggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaggccagcccgggactagaagtgctctgagtgacggggtccagagtatctggacttaaagacacaggcttaaggacgaggtgggaggtagtcagaactgcggctgtctccccaggtgaagaccttgctgctaaatgctgctcagaacaccatggagccacccctgaccccgcccagcccagcaggtgagaagcatcaggcatccccagcccgactcctctgactcctcacagaggtctcttctccttcaggacctctgaaggaggcctccctttctctaccctcagccccgtccatcacccctcatggtcctgtctgtcgcttaccttgggagaaaggcagtgttcaggtgtccatgtccccacccaactctggaggtaaatgacatgtctgtatgtgtgtccccctaagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgaggtgggttggcctgtgccctgccccctccccagcctcctttcccgatctgagtccaggtgcctccttggccccagggctcccgagcacatgccctaaccatgactcagacctcattcctctgtcttctcagctggctggcggggacctggcaggtctactggaggccctgtctgacatgggcctagaggagggagtgaggctgctgaggggtccagaaacccgagacaagctgcccagcacaggtaaaggggcctccctggaaggtggatctggacctggagggccggaggcctgaggctttgactatcccattcctgtccccatttacccccagcagaggtgaaggaagacagtgcgtacgggagccagtcagtggagcaggaggcagagaagctgggcccaccccctgagccaccaggagggctctgccacgggcacccccagcctcaggtgcactgacctgctgcctgcccccagcccccttcccggaccccctgtacagcgtccccacctatttcaaatcttatttaacaccccacacccacccctcagttgggacaaataaaggattctcatgggaaggggaggacccctccttcccaacttatggca

SEQ ID NO: 22 - DNA, 41 bp, artificial
  oligonucleotide or target sequence
gcacttacctacctcaaaaggtgctgcgaaacgttaagtgc

SEQ ID NO: 23 - DNA, 15 bp, Artificial Sequence
  oligonucleotide or target sequence
agatttcgattagac

SEQ ID NO: 24 - DNA, 16 bp, Artificial Sequence
  oligonucleotide or target sequence
tagaattgaagttaaa

SEQ ID NO: 25 - DNA, 14 bp, Artificial Sequence
  oligonucleotide or target sequence
ataactgtgttttc